(19) United States
(12) Patent Application Publication
(10) Pub. No.: US 2025/0136914 A1
(43) Pub. Date: May 1, 2025

(54) SYSTEMS AND METHODS FOR BIOPROCESSING

(71) Applicant: MicrofluidX Ltd, Stevenage (GB)

(72) Inventors: Cesare M. CEJAS, Edgware (GB); Antonio DE GRAZIA, St. Albans (GB); Manjari GHANSHYAM, Hatfield (GB); Sreedhar MAREDDY, Chelmsford (GB)

(21) Appl. No.: 18/679,196

(22) Filed: May 30, 2024

Related U.S. Application Data
(63) Continuation of application No. PCT/GB2022/053051, filed on Dec. 1, 2022.
(60) Provisional application No. 63/285,066, filed on Dec. 1, 2021.
(60) Provisional application No. 63/285,062, filed on Dec. 1, 2021.
(60) Provisional application No. 63/409,983, filed on Sep. 26, 2022.

(51) Int. Cl. C12M 1/00; C12M 1/26; C12N 5/0783

(52) U.S. Cl. CPC C12M 29/10; C12M 29/04; C12M 33/04; C12N 5/0636

(57) ABSTRACT

The present disclosure provides systems and methods for bioprocessing. The systems and methods can be implemented using a cassette comprising a feeding input layer and a bioprocessing layer. The bioprocessing layer can comprise one or more bioprocessing chambers fluidically connected to the feeding input layer. The cassette can permit a fluid to flow from the feeding input layer through the one or more bioprocessing chambers.

### CROSS REFERENCE

This application is a continuation of International Patent Application No. PCT/GB2022/053051, filed Dec. 1, 2022, which claims priority to 63/285,066, filed Dec. 1, 2021, U.S. Provisional Patent Application No. 63/285,062, filed on Dec. 1, 2021, and U.S. Provisional Patent Application No. 63/409,983, filed on Sep. 26, 2022, all of which are incorporated herein by reference in their entireties.

### BACKGROUND

There are various limitations with conventional cell therapy bioprocessing, which can be complex since they can involve (a) the extraction of cells from the patient, (b) the treatment and modification of these cells ex vivo, and then the (c) reintroduction of these cells back inside the body of the patient. High-throughput cell processing destined for cell therapy treatments can involve precise control of flow, collection, growth, and manipulation of biological cells. An aspect to high-throughput cell processing is streamlining the processes involved from seeding all the way to harvesting, with minimal invasive interventions. There is a need of a high-throughput multifunctional microfluidic chips capable of carrying out in situ cell processing without invasive interventions, which can limit the risk of cell loss and contamination that would otherwise accrue with multiple transfers.

### SUMMARY

The present disclosure provides a multifunctional microfluidic-based system that permits streamlined non-invasive in situ bioprocessing operations for adherent and suspension cells. The system can comprise one or more microfluidic chips and micro-fluidics to miniaturize culture devices, increase throughput, and couple with automation scale-ups.

The one or more microfluidic chips (solid supports) can be designed to optimize the bioprocessing operations involved in cell production for cell therapy applications. These operations aimed for cell production can include, for example, seeding, activation, viral or non-viral transduction, proliferation and/or differentiation, washing and/or purification, sampling, and harvesting—all of which can be performed within the bioprocessing chamber of the chip without the need of external transplants and/or invasive interventions.

In an aspect, provided herein is a cassette comprising: (a) a branched network of primary feeding input channels in a first plane and a branched network of primary feeding input channels in a second plane, wherein the first plane is substantially parallel to the second plane; and (b) a bioprocessing layer comprising one or more bioprocessing chambers fluidically connected to (i) the branched network of primary feeding input channels in the second plane and (ii) the branched network of primary feeding input channels in the first plane via the branched network of primary feeding input channels in the second plane, wherein the cassette permits a fluid to flow from the branched network of primary feeding input channels in the first plane, through the branched network of primary feeding input channels in the second plane and through the one or more bioprocessing chambers.

In some cases, the one or more bioprocessing chambers comprise at least 4, 8, 16, 32, 64, 128, or 256 bioprocessing chambers.

In some cases, the one or more bioprocessing chambers are fluidically connected with one another.

In some cases, the cassette further comprises a plurality of connecting input channels connecting the branched network of primary feeding input channels in the first plane and the branched network of primary feeding input channels in the second plane.

In some cases, the cassette permits the fluid to flow from a connecting input channel of the plurality of connecting input channels to a subset of the one or more bioprocessing chambers.

In some cases, a length dimension of the connecting input channels is orthogonal to a length dimension of the branched network of primary feeding input channels in the first plane.

In some cases, each bioprocessing chamber of the one or more bioprocessing chambers comprises a volume of less than 15 mL, 10 mL, 7 mL, 5 mL, 4 mL, 3 mL, 2 mL, 1 mL, or 0.5 mL.

In some cases, each bioprocessing chamber of the one or more bioprocessing chambers comprise a cell culturing surface of less than 300 cm², 200 cm², 100 cm², 90 cm², 80 cm², 70 cm², 60 cm², 50 cm², 40 cm², 30 cm², 20 cm², 10 cm², 6 cm², 5 cm², or 1 cm².

In some cases, the branched network of primary feeding input channels in the first plane comprises symmetrical branched channels with an axis of symmetry about an upstream-most split point.

In some cases, the branched network of primary feeding input channels in the first plane comprises at least 1, 2, 3, 4, 5, 6, 7, 8, 9, 10, 20, 30, 40, 50, or 60 split points.

In some cases, the branched network of primary feeding input channels in a second plane comprises symmetrical branched channels with an axis of symmetry about an upstream most split point.

In some cases, the branched network of primary feeding input channels in a second plane comprises at least 1, 2, 3, 4, 5, 6, 7, 8, 9, 10, 20, 30, 40, 50, or 60 split points.

In some cases, the branched network of primary feeding input channels in the first plane is above the bioprocessing layer.

In some cases, the branched network of primary feeding input channels in the first plane is below the branched network of primary feeding input channels in the second plane.

In some cases, the branched network of primary feeding input channels in the first plane is above the branched network of primary feeding input channels in the second plane.

In some cases, the cassette further comprises a branched network of primary feeding input channels in a third plane fluidically connected to the branched network of primary feeding input channels in the first plane and a branched network of primary feeding input channels in the second plane, wherein the first plane, second plane, and third plane are substantially parallel.

In some cases, the method further comprises a feeding input, and wherein the cassette permits the fluid to flow from the feeding input, through the branched network of primary feeding input channels in the first plane, through the branched network of primary feeding input channels in the second plane and through the plurality of bioprocessing chambers.

In some cases, the branched network of primary feeding input channels in the first plane comprises binary trees.

In some cases, the branched network of primary feeding input channels in the second plane comprises binary trees.

In some cases, the one or more bioprocessing chambers are fluidically connected to each other in parallel.

In some cases, the cassette further comprises a branched network of secondary feeding input channels fluidically connected to, and between, the primary feeding input channels in the second plane and the one or more bioprocessing chambers.

In some cases, the cassette further comprises a branched network of primary feeding output channels, wherein the cassette permits the fluid to flow from the branched network of primary feeding input channels in the first plane, the branched network of primary feeding input channels in the second plane and through the one or more bioprocessing chambers, and the branched network of primary feeding output channels.

In some cases, the cassette further comprises a branched network of secondary feeding output channels fluidically connected to, and between, the one or more bioprocessing chambers and the branched network of primary feeding output channels.

In some cases, the cassette further comprises a plurality of filters, wherein the cassette permits the fluid to flow from the branched network of primary feeding input channels in the first plane, through the branched network of primary feeding input channels in the second plane, through the plurality of bioprocessing chambers, across the plurality of filters, and through the branched network of primary feeding output channels.

In some cases, the cassette further comprises a feeding input connected to the branched network of primary feeding input channels in a first plane.

In some cases, the cassette further comprises a plurality of collection drains fluidically connected to the one or more bioprocessing chambers.

In some cases, the plurality of collection drains comprises collection channels.

In some cases, the cassette further comprises a branched network of primary collection channels in a fourth plane and a branched network of primary collection channels in a fifth plane fluidically connected to the plurality of collection drains, wherein the first plane, the second plane, the fourth plane, and the fifth plane are substantially parallel.

In some cases, a length dimension of the plurality of collection drains is not orthogonal to a length dimension of the branched network of primary collection channels in the fifth plane.

In some cases, the cassette further comprises a branched network of secondary collection channels fluidically connected to, and between, the one or more bioprocessing chambers and the branched network of primary collection channels in the fifth plane.

In some cases, the cassette further comprises a collection hole fluidically connected to the primary collection channels in a fifth plane.

In some cases, the cassette comprises a material that is transparent.

In some cases, the cassette does not comprise a valve.

In some cases, the one or more bioprocessing chambers comprises a biocompatible material.

In some cases, the biocompatible material is a U.S. Pharmacopeia Convention (USP) Class VI material.

In some cases, the cassette permits a substantially equal pressure drop across each bioprocessing chamber of the one or more bioprocessing chambers when the fluid is flowed from the branched network of primary feeding input channels in the first plane, through the branched network of primary feeding input channels in the second plane and through the one or more bioprocessing chambers.

In some cases, the branched network of primary feeding input channels in the first plane, the branched network of primary feeding input channels in the second plane, and the one or more bioprocessing chambers comprise a fluid.

In some cases, the one or more bioprocessing chambers are sterile.

In some cases, the one or more bioprocessing chambers comprise a plurality of cells.

In some cases, each bioprocessing chamber of the one or more bioprocessing chambers comprise at least 0.35, 0.5, 1, 3.5, 5, 10, 15, or 20 million cells/mL.

In some cases, each bioprocessing chamber of the one or more bioprocessing chambers comprises at least 0.25, 0.5, or 1 million cells.

In some cases, the one or more bioprocessing chambers comprise at least 0.05 million, 0.25 million, 0.5 million, 1 million, 10 million, 50 million, 100 million, 150 million, 500 million, 1 billion, or 5 billion cells.

In another aspect, provided herein is a cassette comprising: (a) a plurality of cassette structures defining a cassette flow path volume from a feeding input to a feeding output, the plurality of cassette structures comprising: the feeding input; a branched network of primary feeding input channels in a first plane; a branched network of primary feeding input channels in a second plane, wherein the first plane is substantially parallel to the second plane; one or more bioprocessing chambers, a branched network of primary feeding output channels; the feeding output; and a plurality of feeding input structures defining a feeding input flow path volume from the feeding input to the one or more bioprocessing chambers, the plurality of feeding input structures comprising: the feeding input; the branched network of primary feeding input channels in the first plane; and the branched network of primary feeding input channels in the second plane; wherein the feeding input flow path volume is 30%, 20%, or 10% or less of the cassette flow path volume, wherein the cassette flow path volume corresponds to a total volume of the feeding input channels and a total bioprocessing volume associated with the plurality of cassette structures.

In some cases, the plurality of cassette structures further comprises a branched network of primary feeding input channels in a third plane fluidically connected to the branched network of primary feeding input channels in the first plane and the branched network of primary feeding input channels in the second plane, wherein the first plane, second plane, and third plane are substantially parallel; and the plurality of feeding input structures comprises the branched comprises a branched network of primary feeding input channels in a third plane.

In some cases, the branched network of primary feeding input channels in the first plane and the branched network of primary feeding input channels in the second plane comprise microfluidic channels.

In some cases, the one or more bioprocessing chambers comprise at least 4, 8, 16, 32, 64, 128, or 256 bioprocessing chambers.

In some cases, the one or more bioprocessing chambers are fluidically connected with one another.

In another aspect, provided herein is a cassette comprising: (a) a feeding input and a branched network of primary feeding input channels; (b) a feeding output and a branched network of primary feeding output channels; and (c) a bioprocessing layer comprising one or more bioprocessing chambers, wherein the cassette permits a fluid to flow from the feeding input, through the branched network of primary feeding input channels, through the one or more bioprocessing chambers, through the branched network of primary feeding output channels, and to the feeding output, wherein the cassette provides a ΔP_{cassette}/ΔP_{bioprocessing chamber} that is less than or equal to (1/22)*(n)+6, wherein n is a number of the one or more bioprocessing chambers.

In some cases, n is at least 16.

In some cases, n is at least 32, 64, 128, or 256.

In some cases, a pump is fluidically connected to the cassette.

In some cases, a reagent vessel is fluidically connected to the pump and the cassette.

In some cases, an air vessel comprising CO₂ is fluidically connected to the reagent vessel.

In some cases, a concentration of CO₂ in the air vessel is about 5% or 8% in volume.

In some cases, a de-bubbler is fluidically connected to the reagent vessel and to the cassette.

In some cases, the reagent vessel is separated from the de-bubbler by a first valve and the de-bubbler is separated from the cassette by a second valve.

In some cases, an output vessel is fluidically connected to the cassette.

In some cases, a flow-through sensor is connected to the cassette.

In some cases, a computer is electrically connected to the flow-through sensor.

In some cases, the system further comprises an imaging device.

In some cases, the system is automated.

In some cases, an agitator is coupled to the cassette.

In another aspect, provided herein is a method comprising providing a cassette; and flowing the fluid from the branched network of primary feeding input channels in the first plane, through the branched network of primary feeding input channels in the second plane and through the one or more bioprocessing chambers.

In some cases, each bioprocessing chamber of the one or more bioprocessing chambers comprises a fluid inlet, and wherein a flow rate of the fluid at the inlet of each bioprocessing chamber of the one or more bioprocessing chambers is from about 10 μL/hr×a number of the one or more bioprocessing chambers to about 10 mL/min×a number of the one or more bioprocessing chambers.

In some cases, fluid comprises a plurality of solid particles.

In some cases, a flow rate of the fluid comprising a plurality of solid particles through the branched network of primary feeding input channels in the first plane and the branched network of primary feeding input channels in the second plane is greater than a settling or sedimentation velocity of the plurality of solid particles.

In some cases, the solid particles comprise cells.

In some cases, the solid particles comprise at least 20,000, 200,000, 350,000, 500,000, 1,000,000, 3,500,000, 10,000,00, 25,000,000, or 50,000,000 cells/mL.

In some cases, the cells comprise microorganisms, mammalian cells, HEK293 cells, T-cells, Jurkat cells, CHO cells, mesenchymal stem cells, embryonic stem cells, induced pluripotent stem cells, or hematopoietic stem cells.

In some cases, the method further comprises distributing a substantially equal amount of the cells to each bioprocessing chamber of the one or more bioprocessing chambers to provide distributed cells.

In some cases, the distributing occurs within 5 minutes.

In some cases, the method further comprises washing the distributed cells.

In some cases, the method further comprises expanding the distributed cells to generate expanded cells.

In some cases, the expanding comprises expanding the distributed cells at least 2-fold, 3-fold, 4-fold, 5-fold, 6-fold, 6.5-fold, 7-fold, 8-fold, 9-fold, 10-fold, 11-fold, 12-fold, 25-fold, 50-fold, 100-fold, 150-fold, or 200-fold.

In some cases, the expanding occurs over at least 24 hr, 48 h, 72 hr, 96 hr, 120 hr, 144 hr, 168 hr, 192 hr, 216 hr, 240 hr, 264 hr, 288 hr, 312 hr, 336 hr, 360 hr, 720 hr, 1080 hr, or 1200 hr.

In some cases, the method further comprises imaging the distributed cells.

In some cases, the method further comprises imaging the expanded cells.

In some cases, the method further comprises, using a computer system to predict time to confluence of the expanded cells.

In some cases, the method further comprises contacting the distributed cells with a reagent after the washing.

In some cases, the method further comprises after the contacting, performing a second wash of the distributed cells.

In some cases, the method further comprises after the second wash of the distributed cells, harvesting the distributed cells.

In some cases, a pressure at a fluid input of the cassette is 3 bar or less, 2 bar or less, 1 bar or less, 0.75 bar or less, 0.5 bar or less, 0.25 bar or less, or 0.1 bar or less.

In another aspect, provided herein is a cassette, comprising: (a) a feeding input, a branched network of primary feeding input channels in a first plane, and a branched network of primary feeding input channels in a second plane; (b) a bioprocessing layer comprising one or more bioprocessing chambers; (c) a branched network of primary feeding output channels and a feeding output; (d) a plurality of filters; and (e) a collection output, a plurality of collection drains, a branched network of primary collection channels in a third plane, and a branched network of primary collection channels in a fourth plane, wherein the first plane, the second plane, the third plane, and the fourth plane are substantially parallel, wherein the cassette permits a fluid to flow in a flow path comprising, in order, the feeding input, the branched network of primary feeding input channels in the first plane, the branched network of primary feeding input channels in the second plane, the one or more bioprocessing chambers, the plurality of filters, the branched network of primary feeding output channels, and the feeding output.

In an aspect, provided herein is a method, comprising providing a cassette and flowing a fluid through the flow path.

In some cases, the fluid comprises cells.

In some cases, the method further comprises depositing the cells in the one or more bioprocessing chambers.

In some cases, the cells are not deposited in the branched network of primary feeding input channels in the first plane or the branched network of primary feeding input channels in the second plane.

In some cases, the plurality of filters prevent the cells from entering the branched network of primary feeding output channels.

In some cases, the cassette comprises a second flow path comprising, in order, the feeding input, the branched network of primary feeding input channels in the first plane, the branched network of primary feeding input channels in the second plane, the one or more bioprocessing chambers, the plurality of collection drains, the branched network of primary collection channels in the third plane, the branched network of primary collection channels in the fourth plane, and the collection output.

In some cases, the one or more bioprocessing chambers comprise cells.

In some cases, the cells comprise at least 0.05 million, 0.25 million, 0.5 million, 1 million, 10 million, 50 million, 100 million, 150 million, 500 million, 1 billion, or 5 billion cells.

In some cases, the method further comprises harvesting the cells through the plurality of collection drains.

In some cases, the harvesting comprises applying a suction to the second flow path.

In some cases, the harvesting comprises harvesting at least 75%, 80%, 85%, 90%, 95%, 96%, 97%, 98%, or 99% of the cells to provide harvested cells.

In some cases, the harvesting occurs in 5 min or less, 1 min or less, 50 seconds or less, 40 seconds or less, 30 seconds or less, 20 seconds or less, 10 seconds or less, or 5 seconds or less.

In some cases, at least 75%, 80%, 85%, 90%, 91%, 92%, 93%, 94%, 95%, 96%, 97%, 98%, or 99% of the harvested cells are viable.

In another aspect, provided herein is a method for culturing one or more cells, comprising providing a cassette, distributing the one or more cells in at least one bioprocessing chamber of the one or more bioprocessing chambers; washing the one or more cells; expanding the one or more cells; contacting the one or more cells with at least one reagent; imaging the one or more cells; and harvesting the one or more cells.

In another aspect, provided herein is a microfluidic system comprising one or more cassettes, wherein the microfluidic system is configured for i) flowing a fluid at a pressure of less than 3 bar, or less than 0.5 bar, and ii) culturing at least 50 million cells, at least 500 million cells, or at least 1 billion cells.

In some cases, the one or more cassettes comprise a branched network of primary feeding input channels in a first plane and a branched network of primary feeding input channels in a second plane, wherein the first plane is substantially parallel to the second plane.

In some cases, the one or more cassettes further comprise a bioprocessing layer comprising one or more bioprocessing chambers fluidically connected to (i) the branched network of primary feeding input channels in the second plane and (ii) the branched network of primary feeding input channels in the first plane via the branched network of primary feeding input channels in the second plane.

In some cases, the one or more cassettes permit the fluid to flow from the branched network of primary feeding input channels in the first plane, through the branched network of primary feeding input channels in the second plane and through the one or more bioprocessing chambers.

In some cases, the one or more cassettes further comprise a plurality of connecting input channels connecting the branched network of primary feeding input channels in the first plane and the branched network of primary feeding input channels in the second plane.

In some cases, the one or more cassettes permit the fluid to flow from a connecting input channel of the plurality of connecting input channels to a subset of the one or more bioprocessing chambers.

In some cases, the one or more bioprocessing chambers are separated by a liquid-permeable membrane.

In some cases, the liquid-permeable membrane is impermeable to cells.

In another aspect, provided herein is a cassette comprising (a) a branched network of feeding input channels in a first plane; (b) a bioprocessing layer in a second plane comprising one or more bioprocessing chambers fluidically connected to the branched network of feeding input channels in the first plane; and (c) a branched network of feeding output channels fluidically connected to the one or more bioprocessing chambers, wherein the cassette permits a fluid to flow from the branched network of feeding input channels in the first plane, through the one or more bioprocessing chambers in the second plane, and through the branched network of feeding output channels, and wherein the one or more bioprocessing chambers are fluidically connected with each other.

In some cases, the one or more bioprocessing chambers comprise at least 4, 8, 16, 32, 64, 128, or 256 bioprocessing chambers.

In some cases, the one or more bioprocessing chambers are separated by a liquid-permeable membrane.

In some cases, the liquid-permeable membrane is impermeable to cells.

In some cases, each bioprocessing chamber of the one or more bioprocessing chambers comprises a volume of less than 15 mL, 10 mL, 7 mL, 5 mL, 4 mL, 3 mL, 2 mL, 1 mL, or 0.5 mL.

In some cases, each bioprocessing chamber of the one or more bioprocessing chambers comprise a cell culturing surface of less than 300 cm², 200 cm², 100 cm², 90 cm², 80 cm², 70 cm², 60 cm², 50 cm², 40 cm², 30 cm², 20 cm², 10 cm², 6 cm², 5 cm², or 1 cm².

In some cases, the branched network of feeding input channels in the first plane comprises symmetrical branched channels with an axis of symmetry about an upstream-most split point.

In some cases, the branched network of feeding input channels in the first plane comprises at least 1, 2, 3, 4, 5, 6, 7, 8, 9, 10, 20, 30, 40, 50, or 60 split points.

In some cases, the branched network of feeding input channels in the first plane is above the bioprocessing layer.

In some cases, the branched network of feeding output channels is above the bioprocessing layer.

In some cases, the branched network of feeding output channels is in the first plane.

In some cases, the branched network of feeding input channels in the first plane comprises binary trees.

In some cases, branched network of feeding output channels comprises binary trees.

In some cases, the one or more bioprocessing chambers are fluidically connected to each other in parallel.

In some cases, the cassette comprises a plurality of filters, wherein the cassette permits the fluid to flow from the branched network of feeding input channels in the first plane, through the plurality of bioprocessing chambers in the second plane, across the plurality of filters, and through the branched network of feeding output channels.

In some cases, a feeding input is connected to the branched network of feeding input channels in a first plane.

In some cases, a plurality of collection drains are fluidically connected to the one or more bioprocessing chambers.

In some cases, the plurality of collection drains comprises collection channels.

In some cases, the cassette comprises a material that is transparent.

In some cases, the cassette does not comprise a valve.

In some cases, the one or more bioprocessing chambers comprises a biocompatible material.

In some cases, the biocompatible material is a U.S. Pharmacopeia Convention (USP) Class VI material.

In some cases, the cassette permits a substantially equal pressure drop across each bioprocessing chamber of the one or more bioprocessing chambers when the fluid is flowed from the branched network of primary input channels in the first plane and through the one or more bioprocessing chambers.

In some cases, the one or more bioprocessing chambers are sterile.

In some cases, the one or more bioprocessing chambers comprise a plurality of cells.

In some cases, each bioprocessing chamber of the one or more bioprocessing chambers comprise at least 0.35, 0.5, 1, 3.5, 5, 10, 15, or 20 million cells/mL.

In some cases, each bioprocessing chamber of the one or more bioprocessing chambers comprises at least 0.25, 0.5, or 1 million cells.

In some cases, the one or more bioprocessing chambers comprise at least 0.05 million, 0.25 million, 0.5 million, 1 million, 10 million, 50 million, 100 million, 150 million, 500 million, 1 billion, or 5 billion cells.

In some cases, the first plane is substantially parallel to the second plane.

In some cases, a cassette further comprises: (a) a branched network of gas input channels in a third plane; (b) a gas layer in a fourth plane fluidically connected to the branched network of as input channels in the third plane; and (c) a branched network of gas output channels in the third plane fluidically connected to the one or more bioprocessing chambers, wherein the cassette permits a gas to flow from the branched network of gas input channels in the third plane, through the gas layer in the fourth plane, and through the branched network of gas output channels in the third plane.

In some cases, the cassette permits the gas, while in the gas layer, to dissolve directly into the fluid in the one or more bioprocessing chambers.

In some cases, the third plane is substantially parallel to the fourth plane.

In some cases, the first plane, second plane, third plane, and fourth plane are all substantially parallel.

In some cases, the third plane is above the fourth plane.

In some cases, the fourth plane is above the first plane, and wherein the first plane is above the second plane.

In some cases, the gas layer and the one or more bioprocessing chambers are not separated by a gas permeable membrane.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates a single chip design, in accordance with some embodiments.

FIG. 2 schematically illustrates an exploded view of a single chip design, in accordance with some embodiments.

FIGS. 3A-3C schematically illustrate various views of a parallelized network of chips, in accordance with some embodiments.

FIGS. 4 and 5 schematically illustrates various layers for a cassette comprising parallel chips, in accordance with some embodiments.

FIG. 6 schematically illustrates an exemplary cassette with a parallelized chip configuration, in accordance with some embodiments.

FIG. 7 illustrates an example of a cassette with a parallel chip configuration, in accordance with some embodiments.

FIG. 8 illustrates channel configurations in a cassette with a parallel chip configuration, in accordance with some embodiments.

FIG. 9 schematically illustrate various examples of a cassette with a parallel chip configuration, in accordance with some embodiments.

FIGS. 10A-10D schematically illustrate performance characteristics for a 2×2 and a 4×4 parallelized array of chips, in accordance with some embodiments.

FIG. 10E schematically illustrates an exemplary experimental set-up for flow tests, in accordance with some embodiments.

FIGS. 11-14 schematically illustrate simulations of fluid flow across cassettes with various network arrays of chips, in accordance with some embodiments.

FIG. 15A schematically illustrates a graph of normalized pressure (i.e., delta Pressure of the entire chip array in the cassette/average delta Pressure of individual chip) as a function of chip number, in accordance with some embodiments.

FIG. 15B schematically illustrates a flow resistance through a chip relative to a flow resistance through a parallelized chip array, in accordance with some embodiments.

FIG. 16A schematically illustrates an exemplary compartment comprising 4 chips in a parallel configuration, in accordance with some embodiments.

FIG. 16B schematically illustrates a plot of input channel volume/total volume as a function of chip number, in accordance with some embodiments.

FIG. 17 schematically illustrates an example of a cassette (chip array) comprising more than one more layer, in accordance with some embodiments.

FIG. 18A illustrates the fill scheme of a cassette with channels of the same width, in accordance with some embodiments.

FIG. 18B shows the percent filled of the chambers of the cassette of FIG. 18A as a function of time.

FIG. 19A illustrates the fill scheme of a cassette where the longer channels are 40% wider than the shorter channels, in accordance with some embodiments.

FIG. 19B shows the percent filled of the chambers of the cassette of FIG. 19A as a function of time.

FIG. 20A illustrates the fill scheme of a cassette where the longer channels are 40% thinner than the shorter channels, in accordance with some embodiments.

FIG. 20B shows the percent filled of the chambers of the cassette of FIG. 20A as a function of time.

FIG. 21A illustrates the fill scheme of a cassette with channels of the same resistance, in accordance with some embodiments.

FIG. 21B shows the percent filled of the chambers of the cassette of FIG. 21A as a function of time.

FIG. 22A illustrates the fill scheme of a cassette with channels of the same area, in accordance with some embodiments.

FIG. 22B shows the percent filled of the chambers of the cassette of FIG. 22A as a function of time.

FIG. 23 schematically illustrates a computer system that is programmed or otherwise configured to implement methods provided herein.

FIGS. 24A-24D schematically illustrate a 64-chip array based on four 16-chip units. FIG. 25 schematically illustrates various views of a 64-chip array based on four 16-chip units.

FIGS. 26A-26D schematically illustrates a 64-chip array based on two 32-chip units.

FIG. 27 schematically illustrates various views of a 64-chip array based on two 32-chip units.

FIGS. 28A and 28B illustrate various views of a 32-chip unit.

FIG. 29 schematically illustrates a plot of a percentage of dead volume in a microfluidic device comprising a parallelized chip array as a function of a number of chips in the parallelized chip array.

FIG. 30 schematically illustrates a portion of a parallelized chip array, in accordance with some embodiments.

FIG. 31 schematically illustrates various ports and channels of a microfluidic device prior to passing cells through the device.

FIG. 32 schematically illustrates the outlet holes of the bioprocessing chamber of the microfluidic device after flushing.

FIG. 33 schematically illustrates the channels of the microfluidic chip device after flushing.

FIG. 34 schematically illustrates a table showing the cell counts for the cells collected from the microfluidic device after the channels of the microfluidic device are flushed.

FIG. 35 schematically illustrates an example of a 32-chip cassette comprising more than one more layer, in accordance with some embodiments.

FIG. 36 schematically illustrates another example of a 32-chip cassette comprising more than one more layer, in accordance with some embodiments.

FIG. 37 schematically illustrates an example of a cassette (chip array) comprising more than one more layer and detachable filters integrated with the inlet and outlet channels, in accordance with some embodiments.

FIG. 38 illustrates an example of a cassette (chip array) comprising more than one more layer and a detachable Y-shaped filter integrated with the outlet channel, in accordance with some embodiments.

FIG. 39 schematically illustrates a 32-chip cassette with fused chambers, in accordance with some embodiments.

FIG. 40 schematically illustrates a 32-chip cassette with chambers separated by liquid permeable walls, in accordance with some embodiments.

FIG. 41 schematically illustrates an example of a 64-chip one row cassette with one fused chamber, in accordance with some embodiments.

FIGS. 42A and 42B schematically illustrate an example of a cassette with parallel array of chips in which the chips are fused, in accordance with some embodiments.

FIG. 43 schematically illustrates an example of a 32-chip cassette with fused chambers divided into two compartments down the middle, in accordance with some embodiments.

FIG. 44 schematically illustrates an example of a bifurcated cassette with a single chamber, in accordance with some embodiments.

FIG. 45 schematically illustrates an example of a bifurcated cassette with a single chamber and a gas lid layer, in accordance with some embodiments.

FIG. 46 schematically illustrates an example of a bifurcated cassette with a gas lid layer, a single chamber, and a bioprocessing layer, in accordance with some embodiments.

FIG. 47 schematically illustrates an example of a bifurcated cassette with a single chamber and 50% wider channels, in accordance with some embodiments.

FIG. 48 schematically illustrates an example of a cassette with a 1-to-3 split followed by bifurcated splits and a single chamber, in accordance with some embodiments.

FIG. 49 schematically illustrates an example of a cassette with a 1-to-2 split followed by bifurcated splits with straight channel corners and a single chamber, in accordance with some embodiments.

FIG. 50 schematically illustrates an example of a cassette with a 1-to-2 split followed by bifurcated splits with inclined channels and a single chamber, in accordance with some embodiments.

FIG. 51 schematically illustrates an example of a cassette with a 1-to-2 split followed by bifurcated splits and a single chamber, where the channel outputs from the single chamber contain a filter, and wherein the single chamber has one or more sensors, in accordance with some embodiments.

FIG. 52 schematically illustrates a 32-chip cassette with a single chamber divided into 32 sections by liquid permeable walls, in accordance with some embodiments.

FIG. 53 schematically illustrates a cassette with a gas layer and gas input and outlet channels, in accordance with some embodiments.

### DETAILED DESCRIPTION

While various embodiments have been shown and described herein, it will be obvious to those skilled in the art that such embodiments are provided by way of example only. Numerous variations, changes, and substitutions can occur to those skilled in the art without departing from the disclosure. It should be understood that various alternatives to the embodiments of the disclosure described herein can be employed.

Whenever the term “about,” “at least,” “greater than,” or “greater than or equal to” precedes the first numerical value in a series of two or more numerical values, the term “about,” “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Overview

The present disclosure provides a multifunctional microfluidic-based system that can permit streamlined non-invasive in situ bioprocessing operations for adherent and suspension cells. The system can comprise a microfluidic chip, or a cassette comprising more than one microfluidic chip as described in further detail below.

The one or more microfluidic chips can be designed to optimize the operations involved in cell production for cell therapy applications. The operations for cell production can include, for example, seeding, treatment, proliferation and/or differentiation, washing and/or purification, sampling, and harvesting—all of which can be performed within the bioprocessing chamber (e.g., on a culture surface or within a volume of the bioprocessing chamber) of the microfluidic chip or cassette without the need of external transplants and/or invasive interventions.

### Cell Therapy

The systems and methods of the present disclosure can be used for cell therapy applications. Cell therapy can be a treatment approach in which functional and healthy cells, cultured ex vivo, are administered into or to a subject (e.g., a patient).

The systems of the present disclosure are designed to improve the cell culture process. In one aspect, the present disclosure provides a miniaturized chip comprising a bioprocessing chamber that is capable of performing bioprocessing operations involved in cell culture. The chip can utilize microfluidics, which can involve manipulating fluids inside channel dimensions of the micrometer range. In some embodiments, the channels described herein (including, for instance, feeding input channels, feeding output channels, input harvest channels, output harvest channels, etc.) can have one or more channel dimensions. The one or more channel dimensions can correspond to one or more of a channel width, a channel length, a channel height, or a channel diameter. The channel dimensions can range from about 1 micrometer to about 10 centimeters. In some cases, the channel dimensions can be less than 1 micrometer. In some cases, the chip design extends to the principles of mesofluidics or macrofluidics because certain \channel dimensions can be greater than 1 centimeter and sometimes greater than 10 centimeters. In some cases, the channels described herein (including, for instance, feeding input channels, feeding output channels, input harvest channels, output harvest channels, etc.) can have a channel volume. The channel volume can range from 10% of the total chip volume to 90% of the total chip volume. In some cases, the channel volume can be less than 10% of the total chip volume. In some cases, the channel volume can be greater than 90% of the total chip volume.

Microfluidic cell culture can provide several advantages, including, for instance: (1) better control of process parameters: cells can have equal access to molecules present in the surrounding fluid due to homogenous cell distribution and fluid circulation in microenvironments, which can result in a more homogeneous end product and less process failure (e.g. cell death); (2) better overall cell health: e.g., paracrine effects can be amplified in a small environment (i.e. microscale), which can lead to better cell expansion and phenotype control; (3) a reduction in reactant volume: can be 10-20 fold reduction compared to other conventional bioprocessing systems, due to smaller volumes of fluid used in microfluidic chips as well as the ability to recirculate unspent reactant (e.g., growth media (fluid) can be re-enriched and recirculated at defined intervals, e.g., due to rapid oxygen or glucose depletion inside the chip). As used herein, homogenous cell distribution may refer to the distribution of cells such that a density of cells is approximately uniform across a target area.

### Chip

In some cases, the chip can comprise a microfluidic chip. The chip or microfluidic chip can comprise a bioprocessing chamber. The bioprocessing capabilities of the chip can be scaled up via parallelization to achieve high throughput bioprocessing. A plurality of chips can be parallelized in a cassette. In some cases, a plurality of cassettes can be placed in a machine for parallelized bioprocessing. The plurality of cassettes can be stackable.

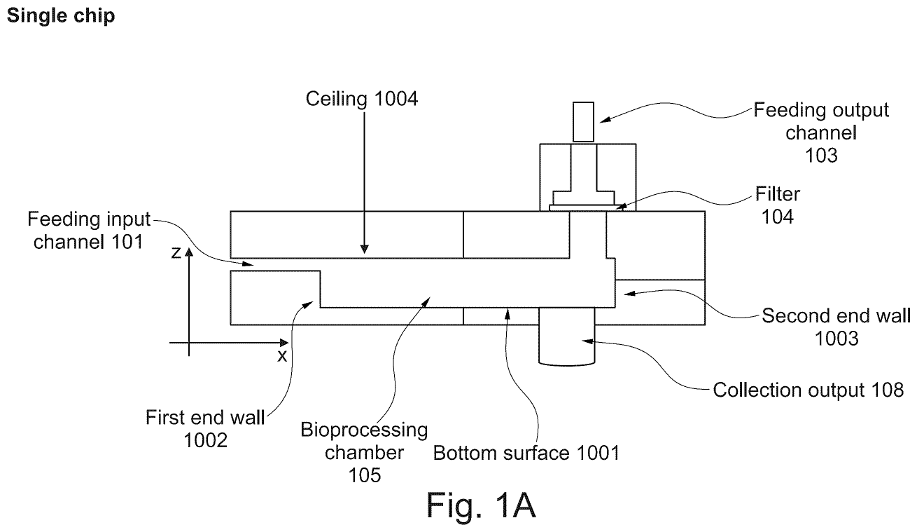

FIG. 1 schematically illustrates an exemplary chip. A chip can be a support or a solid support comprising a bioprocessing chamber, e.g., one bioprocessing chamber. The chip can comprise a feeding input that connects to one or more feeding input channels 101. The feeding input can comprise a single hole or a plurality of holes. The plurality of holes can provide separate inputs for both seeding and perfusion. These feeding input channels can be used for seeding and perfusion. The input channels 101 can take several forms. It can be a single channel or a plurality of channels in the form of a standard or modified binary tree network.

These input channels 101 can feed into a bioprocessing chamber 105, which can comprise a recess in fluid communication with the one or more feeding input channels 101. The recess can comprise a vertical depth perpendicular to the flow direction where cells can settle. The recess can be protected from damaging shear stress because of minimal fluid velocity acting on the recess. The bioprocessing chamber 105 can be elongated in the primary direction of seeding and perfusion flow, such that length>>width. In some cases, the microfluidic chip or the bioprocessing chamber of the microfluidic chip can have a length that is about, at least, or at most 1.5, 2, 3, 4, 5, 6, 7, 8, 9, 10, 20, 30, 40, 50, or 60 times its width. In some embodiments, the edges of the bioprocessing chamber 105 (e.g., at the ends) can be curved to minimize culture dead zones. Having curved edges can also facilitate initial chip wetting as opposed to rigid, sharp edges. In some cases, the curved edges may comprise a fillet or a rounded section that provides a transitional surface between two adjacent edges of the bioprocessing chamber.

In some cases, the bioprocessing chamber can comprise a recess with one or more walls that are angled relative to the feeding input channels and/or the feeding output channels. In some non-limiting embodiments, the angle can range from about 45 degrees to about 90 degrees. In some cases, the bioprocessing chamber can have one or more dimensions. The one or more dimensions can comprise, for example, a length, a width, a height, or a depth. The one or more dimensions of the bioprocessing chamber can range from about 1 millimeter to about 60 centimeters. In some cases, the dimensions of the bioprocessing chamber can be less than 1 millimeter. In some cases, the dimensions of the bioprocessing chamber can be greater than 60 centimeters. The bioprocessing chamber can comprise a volume of less than 15 mL, 10 mL, 7 mL, 5 mL, 4 mL, 3 mL, 2 mL, 1 mL, or 0.5 mL.

In some cases, the bioprocessing chamber can have a bottom surface, as described elsewhere herein. The bottom surface can be used for cell culturing. The bottom surface can have a surface area ranging from about 1 mm² to about 300 cm². In some cases, the surface area can be less than 1 mm². In some cases, the surface area can be greater than 300 cm². The bottom surface can have a surface area of less than 300 cm², 200 cm², 100 cm², 90 cm², 80 cm², 70 cm², 60 cm², 50 cm², 40 cm², 30 cm², 20 cm², 10 cm², 6 cm², 5 cm², or 1 cm².

In some cases, a length dimension of the bioprocessing chamber can be at least 2x, 3x, 4x, 5x, 10x, 15x, or 20x a width dimension of the bioprocessing chamber. In some cases, the bioprocessing chamber has a height of at least 0.1 mm, 0.2 mm, 0.3 mm, 0.4 mm, or 0.5 mm.

The bioprocessing chamber can comprise a cross-sectional shape. The cross-sectional shape can be a circle, an oval, an ellipse, a triangle, a square, a rectangle, or any other polygon having three or more sides. The cross-sectional shape can correspond to a horizontal cross-section and/or a vertical cross-section of the bioprocessing chamber.

Towards the downstream end of the bioprocessing chamber 105, there can be two outlets positioned above and below the bioprocessing chamber 105. The upper outlet can connect to a filter 104 (e.g., a filter membrane) and/or a feeding output channel 103. In some cases, the chip may comprise a plurality of upper outlets connected to one or more filters. The one or more filters may be positioned at or near one or more upper outlets. In some cases, the one or more filters may be positioned in front of or within the one or more upper outlets. In some cases, the chip may comprise a plurality of filters that are stacked on top of each other, or arranged side-by side or in series relative to each other. The plurality of filters may have different shapes, sizes, and/or filtering capabilities. In some cases, one or more filters may be provided in each of a plurality of upper outlets. The filter 104 can serve as a barrier to prevent cells from exiting the chamber prematurely, hence increasing seeding efficiency. During perfusion via the feeding input channel, while growth medium can be replenished, e.g., gradually replenished, by a fresh batch of fluid, the cells can still be retained in the bioprocessing chamber 105 while the fluid exits through the feeding output channel 103. In some cases, the filter 104 can comprise a filter membrane, and the filter membrane can comprise filter materials such as polyethersulfone (PES), polyester track-etched (PETE), cellulose nitrate, polytetafluororthethylene (PTFE) e.g., with a pore size structure of about 5 micrometers. In some cases, the filter 104 comprises a pore size of less than 10 μm, less than 7.5 μm, less than 5 μm, less than 2.5 μm, or less than 1 μm, or less than 0.45 μm. The shape of the filter can be rectangular, oval, elliptical, or circular. In some cases, the shape can comprise any regular or irregular shape. In some cases, the shape can comprise any shape having three or more sides. In some cases, a dimension of the filter can range from about 1 μm to about 50 cm. The dimension can correspond to a length, a width, or a thickness of the filter. In some cases, a single filter strip can be used across the entire bioprocessing chamber. The lower outlet of the chip can be fluidically connected to a drain 108 for harvesting or collection purposes. In some cases, the chip may comprise a plurality of drains disposed at or near a bottom surface of the bioprocessing chamber. In some cases, the chip may comprise a plurality of lower outlets fluidically connected to the plurality of drains. When the outlet is open, fluid containing cells can be drawn out of the bioprocessing chamber 105. To improve harvest efficiency, fluid can be pulled via the lower outlet, e.g., via a syringe pump or a suction generated by a negative pressure source. Alternatively, fluid can be introduced via the feeding input and/or feeding output to help “push” the fluid out. Alternatively, collecting can also be done via a combination of push and pull, where fluid is simultaneously pulled from the lower outlet and pushed via the feeding input and/or output. The collection drain 108 can be positioned directly below the filter 104 or at a position away from the filter 104. In some non-limiting embodiments, an inclined/sloped structure can be provided to facilitate the fluid's exit. In some cases, the inclined/sloped structure can be integrated with the bottom surface of the bioprocessing chamber 105 and can connect the bioprocessing chamber 105 to the drain 108. Alternatively, the inclined/sloped structure can be formed as part of the drain 108. In some cases, the drain may be positioned at or near a bottom portion of one or more walls of the bioprocessing chamber. In some cases, the drain may be located upstream of a feeding output and/or downstream of a feeding input. In some cases, the drain may be positioned to the left of the feeding input or feeding output. In other cases, the drain may be positioned to the right of the feeding input or feeding output.

In some embodiments, the filter can comprise a pore size. The pore size can range from about 1 nanometer to about 1 millimeter. In some cases, the filter can comprise a plurality of different pore sizes ranging from about 1 nanometer to about 1 millimeter.

In some cases, the filter can comprise a membrane. The membrane can be permeable or semi-permeable. The membrane can comprise, for example, Polytetrafluoroethylene (PTFE) or expanded polytetrafluoroethylene (ePTFE), polyethersulfone (PES), modified polyethersulfone (mPES), polysulfone (PS), modified polysulfone (mPS), ceramics, polypropylene (PP), cellulose, regenerated cellulose or a cellulose derivative (e.g., cellulose acetate or combinations thereof), polyolefin, polypropylene, polytetrafluoroethylene, polyvinyl chloride, polyester, or any other type of polymer. In some non-limiting embodiments, the membrane can comprise a biomedical polymer, e.g., polyurethane, polyethylene, polypropylene, polyester, poly tetra fluoro-ethylene, polyamides, polycarbonate, or polyethylene-terephthalate.

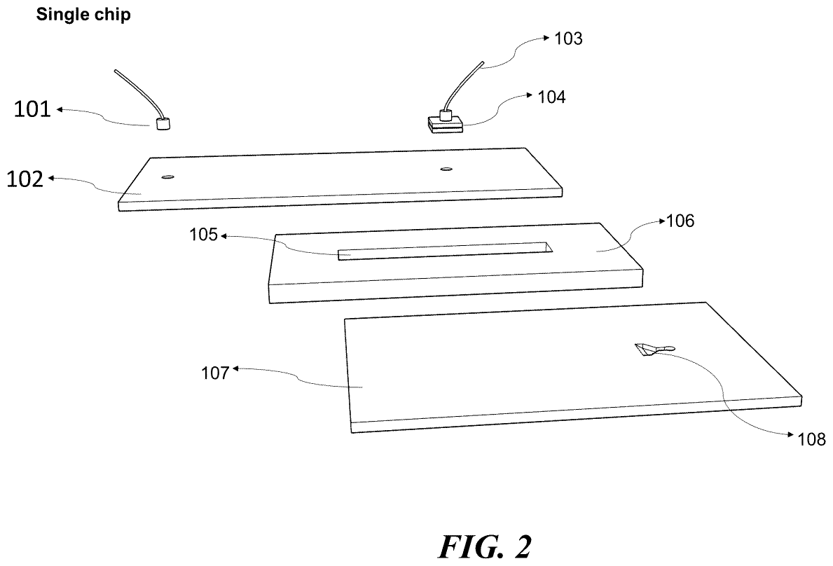

FIG. 2 schematically illustrates an exploded view of the components and layers of the exemplary chip shown in FIG. 1. The chip 100 can comprise one or more feeding input channels 101. The chip can further comprise an upper layer 102 with an aperture for receiving one or more materials (e.g., cells) transported through the one or more feeding input channels 101. The chip can further comprise one or more feeding output channel 103 and a filter 104.

In some embodiments, the chip can comprise a middle layer 106 comprising a bioprocessing chamber 105 that is carved out of the middle layer 106. In some embodiments, the chip can further comprise a bottom layer 107. The bottom layer can comprise a collection/harvest drain 108. In one embodiment, the drain 108 can comprise a circular structure. In some cases, the drain may have a cross-sectional shape that is circular, oval, elliptical, square, or rectangular. In some cases, the drain may have a cross-sectional shape having three or more sides. The cross-sectional shape may correspond to a regular shape or an irregular shape. In some cases, the bottom layer 107 can have an inclined or sloped structure leading to the drain 108 to help facilitate the fluid's exit. In other cases, the inclined or sloped structure can be formed as part of the drain 108 to help facilitate the fluid's exit. In some embodiments, the collection drain 108 can be fluidically connected to the bioprocessing chamber via the bottom surface of the bioprocessing chamber. In some cases, the collection drain 108 may be fluidically connected to the bioprocessing chamber via one or more holes, apertures, channels, or passageways in or through at least a portion of the bottom surface of the bioprocessing chamber.

The cells described herein can comprise a range of sizes. In some cases, the cells can have a size of at least about 1 micrometer, 5 micrometers, 10 micrometers, 20 micrometers, 30 micrometers, 40 micrometers, 50 micrometers, 60 micrometers, 70 micrometers, 80 micrometers, 90 micrometers, 100 micrometers, or any size that is between any of the preceding values. In some cases, the cells can have a size that is less than about 1 micrometer. In some cases, the cells can have a size that is at most about 1 micrometer, 900 nanometers, 800 nanometers, 700 nanometers, 600 nanometers, 500 nanometers, 400 nanometers, 300 nanometers, 200 nanometers, 100 nanometers, 90 nanometers, 80 nanometers, 70 nanometers, 60 nanometers, 50 nanometers, 40 nanometers, 30 nanometers, 20 nanometers, 10 nanometers, or less.

### Materials

The microfluidic device can be fabricated in optically transparent material or a combination of different types of materials. The bioprocessing chamber that can be used for cell culture can be made of a USP (United States Pharmacopoeia) Class VI Material. Such materials can be transparent so that imaging technology can be coupled. The device can also possess tolerances on the design requirements (e.g., channels) not lower than 5 micrometers in absolute value for the smallest feature and 5% for larger dimensions. This can ensure that fabrication of these devices can be suitable with standard manufacturing processes (e.g., sheet or roll processing). The device can also comprise usable surface culture space (for the individual chip) that is potentially capable of handling up to at least about 10 million cells, 20 million cells, 30 million cells, 40 million cells, 50 million cells, 60 million cells, 70 million cells, 80 million cells, 90 million cells, 100 million cells, or more.

The device can have certain favorable properties. For example, the device can favor homogenous distribution or collection of solids (i.e., cells) and prevent premature collection of seeded cells. In some embodiments, seeding efficiency (i.e., the number of cells actually seeded relative to the number of cells provided) can be greater than about 90%, 91%, 92%, 93%, 94%, 95%, 96%, 97%, 98%, or 99%. The device can also be versatile for adherent and suspension solids (i.e., cell culture). The fluid flow coming from perfusion can avoid generating high shear stress that would potentially damage the cells. For suspension cells, the flow can circulate and exit the chip without flushing the cells. The device can also favor cell growth with minimal invasion and favor sampling procedures without invasive procedures. In some cases, the device can favor cell/particle extraction from the device at >90% efficiency with very minimal cells still stuck in the system. In some cases, the device can favor cell/particle extraction from the device at about 50%, 60%, 70%, 80%, or 90% efficiency with very minimal cells still remaining in the system.

The chip can possess usable surface culture area (for the network) of at least 1 cm², which can represent at least about 70% of the total chip footprint.

### Scale Up and Parallelization

The chips of the present disclosure can be designed in a way such that scale-up does not require a massive overhaul of design, i.e., the chip can retain its original features even when parallelized and multiplexed in a cassette.

First, an individual microfluidic chip can be fabricated. Then, the individual chip can be used as a building block towards a more sophisticated parallel array network for high-throughput cell processing. In some cases, the network array can handle up to 3 bars of pressure without leakage. In some cases, the network array can handle pressure of up to about 1 bar, 2 bars, 3, bars, 4 bars or more without leakage. Through parallelization, there can be no significant change in the cell's microenvironment, since in some cases, at every point in the scale-up, the dimensions of the individual chips and the bioprocessing chambers do not change. Rather, a greater number of chips can be added to the array in a controlled manner to manage hydrodynamic resistances.

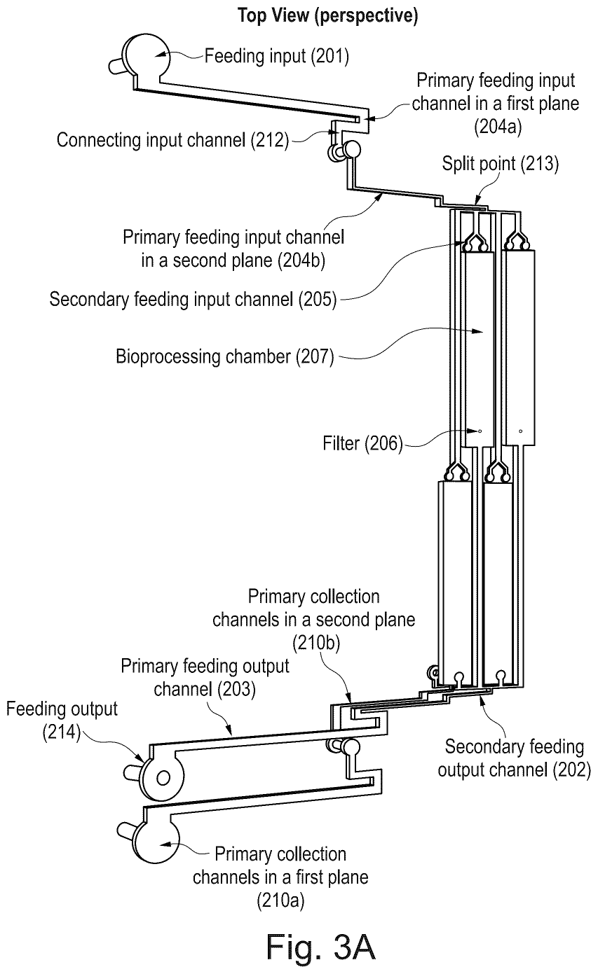

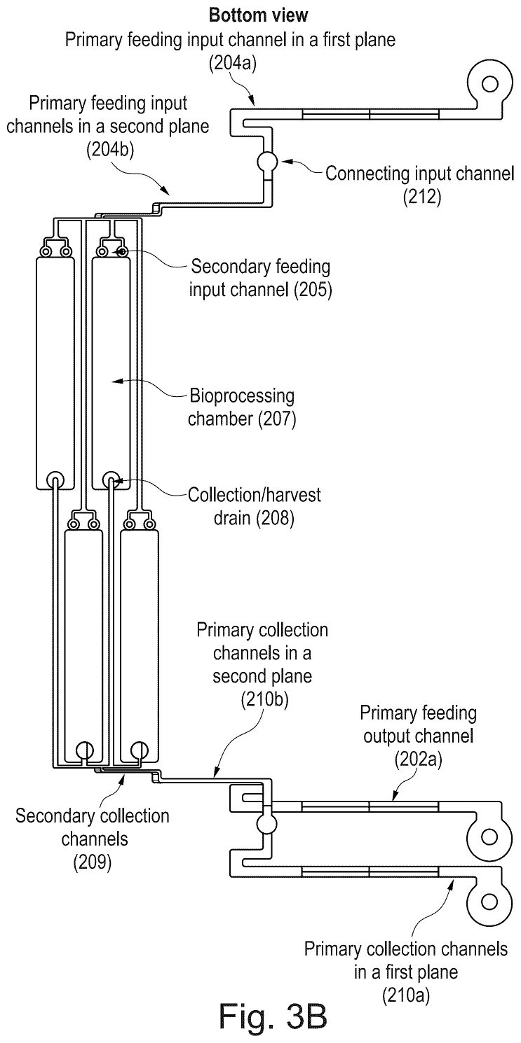

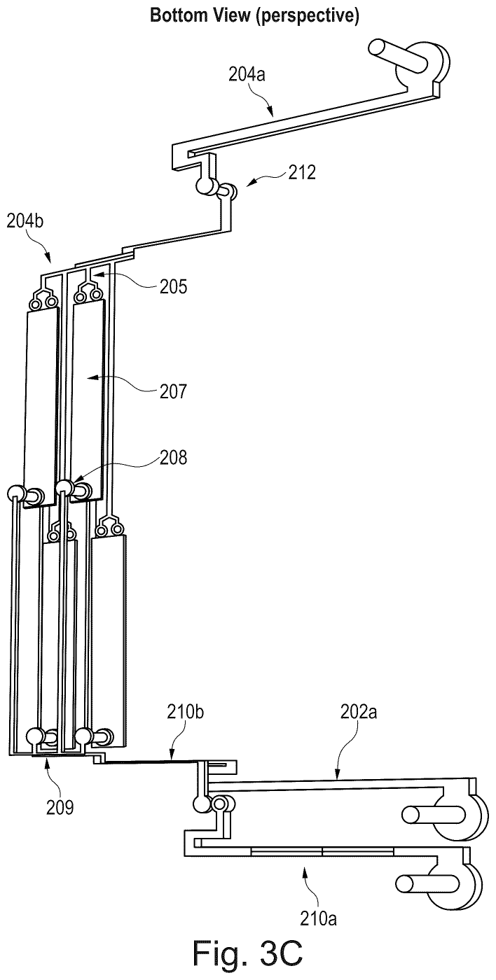

FIGS. 3A-3C schematically illustrates an example of individual chips arranged in a chip network. The chip network can be parallelized. The chips can be placed in a 2×2 array, for instance. The chip array can be fluidically connected to a feeding input 201 via a single primary feeding input channel in a first plane 204a and/or a single primary feeding input channel in a second plane 204b. The single primary feeding input channel in the first plane 204a and the single primary feeding input channel in the second plane 204b can be in fluid communication with each other via a connecting input channel 212. The primary feeding input channels can diverge at a split point 213 to feed the individual chips. The feeding input channel(s) directly leading into the respective bioprocessing chambers 207 of the individual chips can comprise a secondary feeding input channel or a plurality of secondary feeding input channels 205. In some cases, the feeding input channel(s) can comprise a single channel, or a plurality of channels, fluidically connected to each bioprocessing chamber. The primary feeding input channels 204a and/or 204b can be positioned on a different layer than the secondary feeding input channels 205. The bioprocessing chambers 207 can connect to the filter 206 (e.g., filter membrane), e.g., in the downstream end, and then to the secondary feeding output channels 202, which can converge to a primary feeding output channel 203. In some cases, one or more collection drains can be provided in the lower layer beneath the filters, which can converge to one or more primary collection channels. In some embodiments, the one or more primary collection channels can comprise a primary collection channel in a first plane 210a and a primary collection channel in a second plane 210b. The primary feeding output channel 203 can be in fluid communication with a feeding output 214 from which one or more cells can be collected or harvested. Having multiple layers for the different components can help to manage fluid resistances (and pressure drops) by reducing channel length and minimizing changes in channel direction. The multiple layer design can also help to optimize the bioprocessing chamber while increasing the packing density of the chips and reducing dead space or volume.

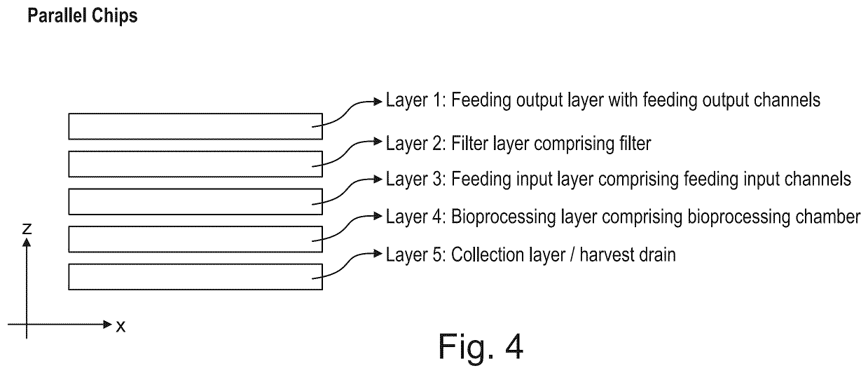

FIG. 4 schematically illustrates a configuration of parallel chips. The configuration can comprise a first layer. The first layer can comprise a feeding output layer with feeding output channels. The configuration can comprise a second layer. The second layer can comprise a filter layer comprising one or more filters (e.g., filter membranes). The configuration can comprise a third layer. The third layer can comprise a feeding input layer comprising feeding input channels. The configuration can comprise a fourth layer. The fourth layer can comprise a bioprocessing layer comprising bioprocessing chambers. The configuration can comprise a fifth layer. The fifth layer can comprise a collection layer/harvest drain.

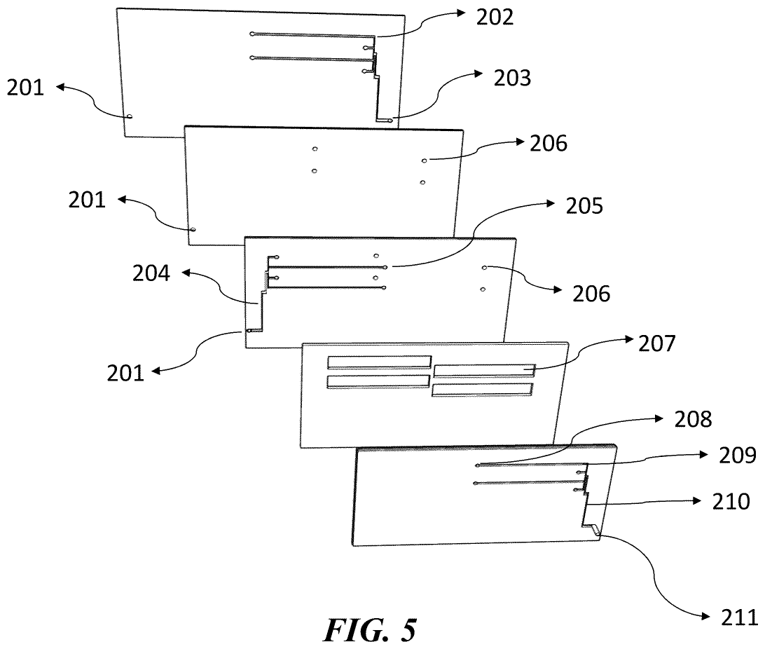

FIG. 5 schematically illustrates a parallelized chip configuration comprising multiple layers. The parallelized chip configuration can comprise a feeding input hole 201 that connects one or more of the multiple layers. The chip configuration can further comprise one or more secondary feeding output channels 202 and one or more filters 206 (e.g., filter membranes). In some cases, the one or more secondary feeding output channels 202 can connect directly to the one or more filters 206.

In some embodiments, the chip configuration can further comprise one or more primary feeding output channels 203. The one or more primary feeding output channels 203 can function as the output of the entire array of parallelized chips.

In some embodiments, the chip configuration can further comprise one or more primary feeding input channels 204. The one or more primary feeding input channels 204 can connect to the feeding input hole 201 and feed the entire array network. In some embodiments, the chip configuration can further comprise one or more secondary feeding input channels 205. The secondary feeding input channels 205 can feed one or more individual chips in the array.

In some embodiments, the chip configuration can further comprise one or more bioprocessing chambers 207. In some embodiments, the chip configuration can further comprise one or more collection or harvest drains 208. The one or more collection or harvest drains 208 can comprise a circular structure. In some cases, an inclined or sloped structure leading to the drain can be utilized to help facilitate the fluid's exit.

In some embodiments, the chip configuration can further comprise one or more secondary collection channels 209. The one or more secondary collection channels 209 can be configured to collect fluid or cells from each individual chip.

In some embodiments, the chip configuration can further comprise one or more primary collection channels 210. The one or more primary collection channels 210 can be configured to collect fluid or cells from the entire array network and can be fluidically connected to at least one collection or harvest hole 211. The one or more primary collection channels 210 can comprise one or more primary collection channels in a first plane and one or more primary collection channels in a second plane. The first plane can be different than the second plane.

In some cases, the one or more secondary feeding output channels 202 and the one or more primary feeding output channels 203 can be located on a same layer of the parallelized chip configuration. In some cases, the one or more secondary feeding output channels 202 and the one or more primary feeding output channels 203 can be on different layers than the one or more filters 206 (e.g., filter membranes). In some cases, the one or more secondary feeding output channels 202, the one or more primary feeding output channels 203, and the one or more filters 206 can be on different layers than the one or more primary feeding input channels 204 and the one or more secondary feeding input channels 205. In some cases, the one or more bioprocessing chambers 207 and the one or more collection/harvest drains 208 can be on different layer than any of the other components or features of the parallelized chip configuration. In some embodiments, the one or more secondary collection channels 209, the one or more primary collection channels 210, and the one or more collection/harvest holes 211 can be provided on a same layer, which layer can be a different layer than the other previously described layers.

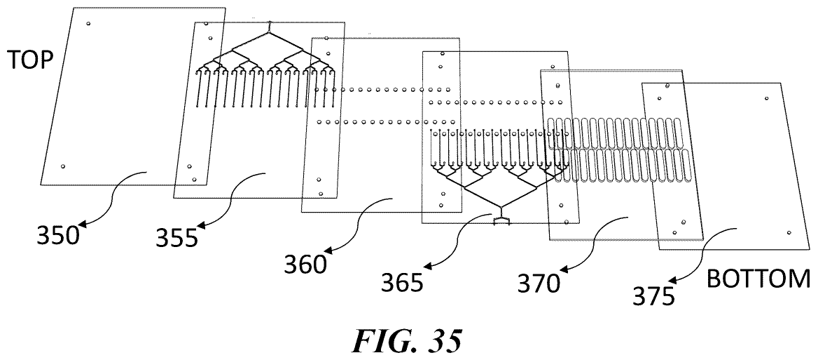

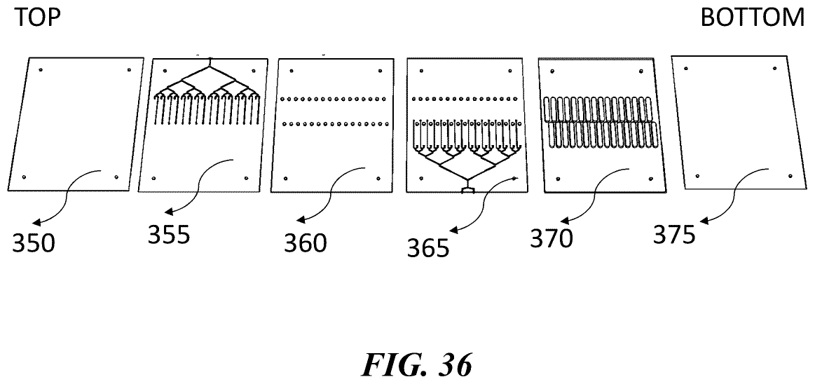

FIGS. 35 and 36 schematically illustrate a parallelized chip configuration comprising a 32-chip cassette with multiple layers. Layer 350 represents the top layer. Layer 350 can be flat and may be made from a cyclic olefin copolymer (COC) or similar compound. Layer 355 may be directly underneath layer 350 and may comprise one or more feeding input channels. Layer 355 can be made from polydimethylsiloxane (PDMS). In some cases, layer 355 is formed via thermal printing of a liquid precursor. Layer 355 may be located directly on top of a hole layer 360. The hole layer 360 can be made from PDMS. In some cases, hole layer 360 is injected molded, comprises imprinted wells, or has laser cut holes. Layer 365 may be located underneath hole layer 360. In some embodiments, layer 365 comprises one or more primary feeding output channels. The one or more primary feeding output channels can function as the output of the entire array of parallelized chips. Layer 365 can be made from PDMS. In some cases, layer 365 is formed via thermal printing of a liquid precursor. Layer 370 may be located underneath layer 365. Layer 370 can comprise one or more bioprocessing chambers. In FIGS. 35 and 36, layer 370 is shown with 32 bioprocessing chambers. Layer 370 can be made from a polymer, such as PMMA, PETG, or COC. A bottom layer 375 can be located underneath layer 370. Layer 375 can be flat and may be made from COC.

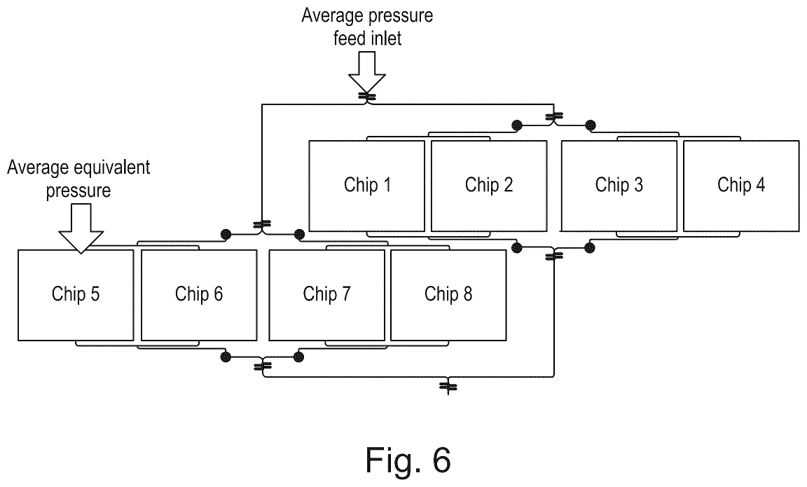

FIG. 6 illustrates a plurality of chips arranged in a parallelized configuration. The parallelized configuration can comprise, in some cases, multiple pairs of parallel chips. The multiple pairs of parallel chips can also be arranged in a parallelized configuration. In some cases, one or more computer processors and/or sensors can be used in combination to modulate the average pressure at the feed inlet to the chips and the average pressure of fluid into or out of the various chips in the parallelized configuration.

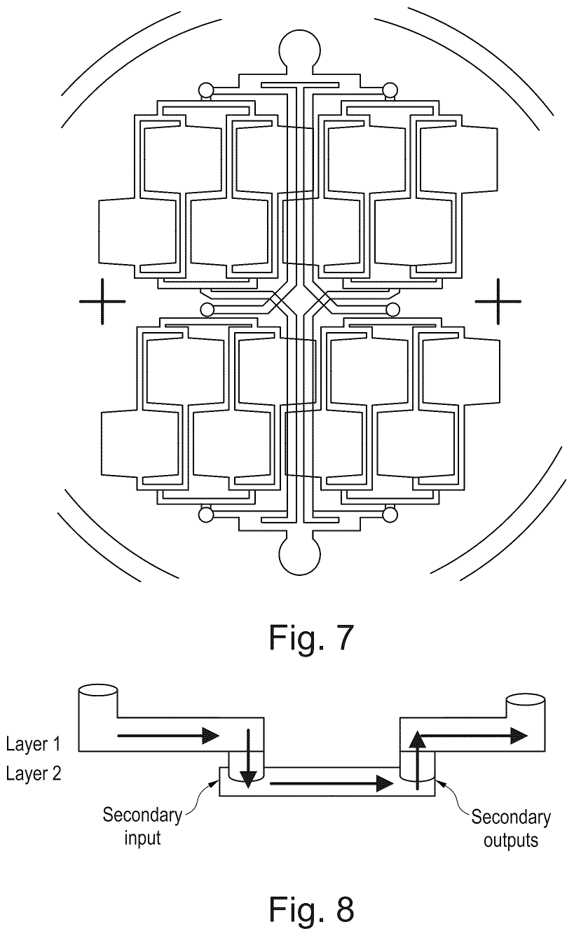

FIG. 7 schematically illustrates an example of a parallel chip configuration, in accordance with some embodiments. Parallelization can be achieved by compartmentalization into smaller groups of chips or separating the main branch into an equal number of smaller branches, each group having a manageable fluidic resistance. These smaller groups can then be integrated into the array via a separate feeding layer, which can minimize the channel length required to reach each group and therefore the fluidic resistance of the array. This can also minimize the footprint of the array, since no additional footprint is needed for the primary feeding channels.

Referring to FIGS. 7 and 8, the parallel chip array can comprise a fluid distribution network on a first layer and a plurality of chips on a second layer. The fluid distribution network can distribute fluid containing one or more cells to the plurality of chips via one or more secondary inputs. In some cases, the plurality of chips can be in fluid communication with an outlet of the fluid distribution network via one or more secondary outputs. In some cases, each compartment can comprise 4, 8, 16, 32, or 64 chips. Fluid entering the secondary output can only see the resistance of 4 chips. This can be achieved via separation of layers: (1) one layer distributes the fluid coming from the main input as well as the fluid going out to the main output; and (2) another layer underneath the first that holds the array.

In some cases, the fluid distribution network can comprise one or more primary input channels and one or more secondary input channels which feed into one or more compartments comprising 4 chips. The fluid distribution network can further comprise one or more primary output channels and one or more secondary output channels. FIG. 9 illustrates an example of one compartment of 4 chips.

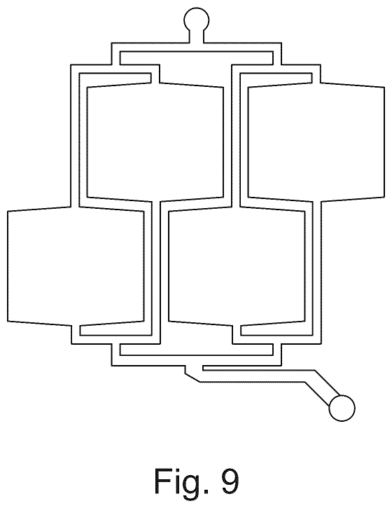

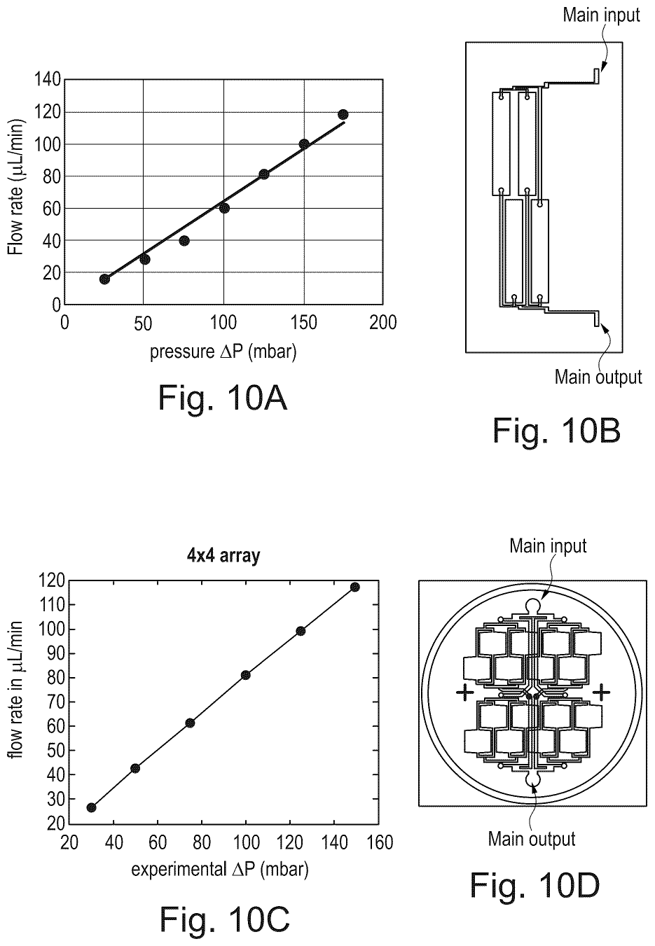

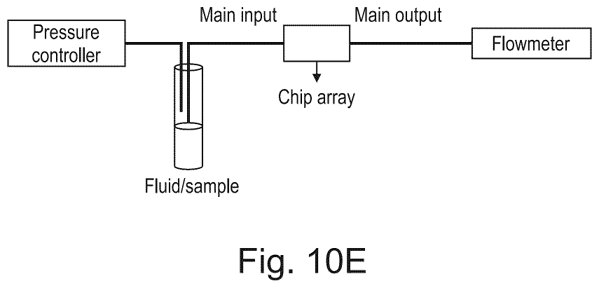

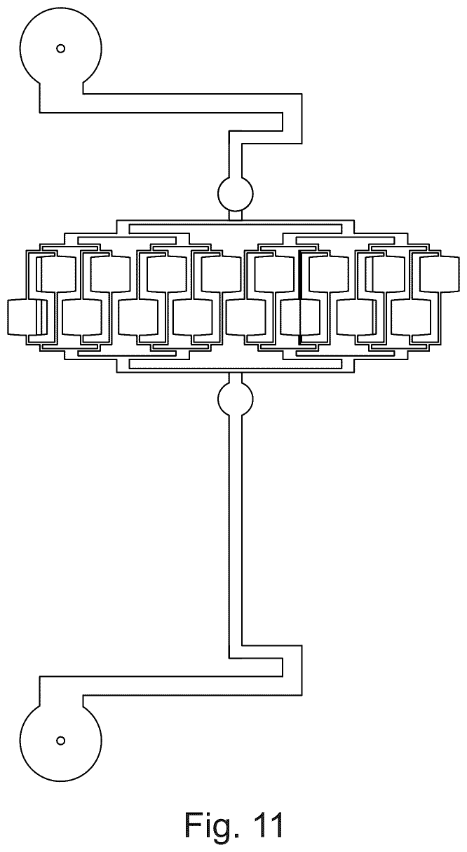

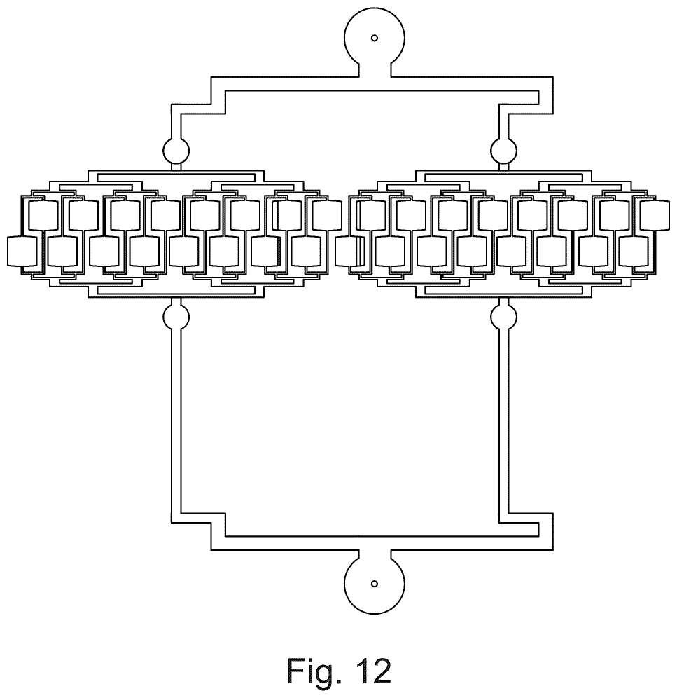

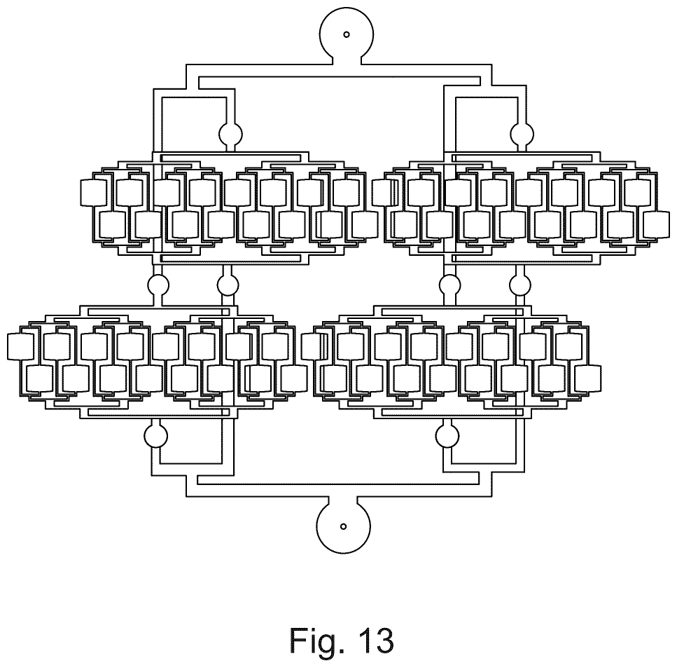

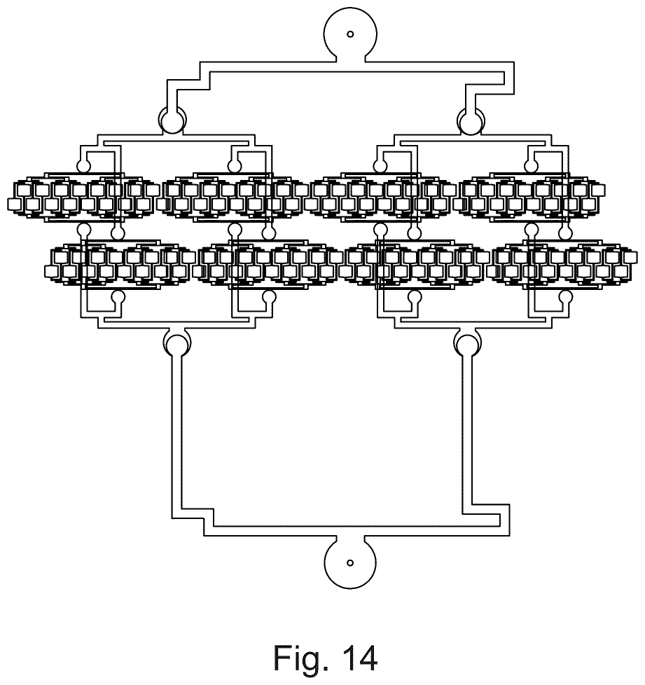

FIG. 10A illustrates flow rates in microliters per minute as a function of delta Pressure in mbar for a 2×2 chip array as shown in FIG. 10B. An increase in flow rate through the 2×2 chip array can be positively correlated with an increase in delta P. FIG. 10C illustrates flow rates in microliters per minute as a function of experimental delta Pressure in mbar for a 4×4 chip array as shown in FIG. 10D. An increase in flow rate through the 4×4 chip array can be positively correlated with an increase in delta P. FIG. 10E illustrates an exemplary experimental set-up for flow tests. The flow tests can be conducted using a pressure controller that pushes fluid at a defined pressure ΔP in mbar. Fluid from the sample container then flows into the chip array via the primary inlet. The fluid exits the chip array via the primary outlet and then flows through the flowmeter, which records the corresponding flow rate (in μL/min). Putting the flow meter at the outlet can provide a general measurement of the overall resistance of the chip array. FIGS. 11-14 illustrate numerical simulations of fluid flow across increasing network arrays of parallelized chips.

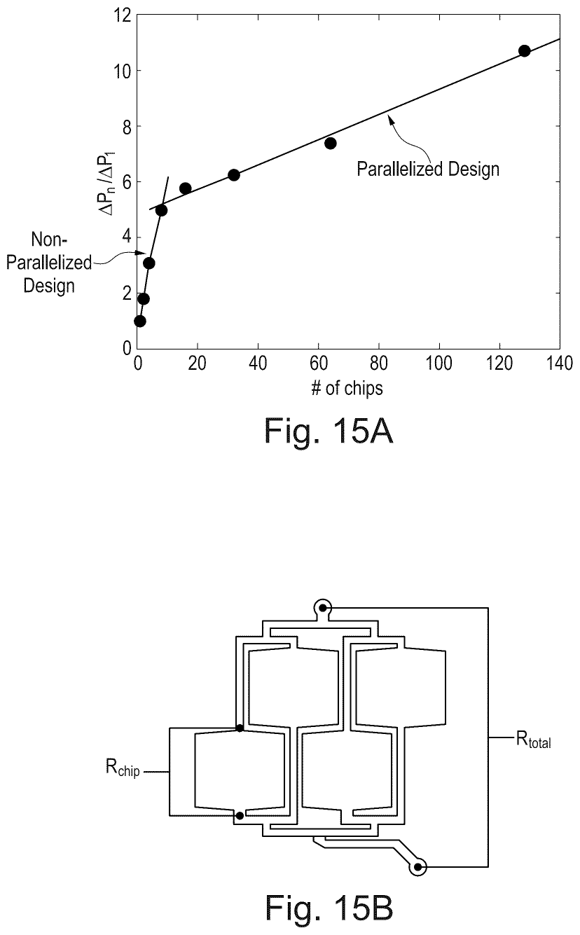

FIG. 15A illustrates a graph of normalized pressure (i.e., delta Pressure of cassette/average delta Pressure of chips) as a function of the number of chips in an array. The normalized pressure can gradually increase by leveraging the parallelization techniques described herein and as the number of chips in a parallelized array increases, as opposed to non-parallelized or single-layer parallelization configurations where the normalized pressure increases steeply.

FIG. 15B illustrates a schematic of an exemplary 4-chip array. The flow resistance across a single chip can be optimized relative to the flow resistance across the entire chip array. The flow resistance (which can be caused by shear stresses due to friction) can directly or indirectly cause a pressure drop as the fluid flows along a flow path through the chips and/or the chip array. In some cases, the pressure drop across a single chip can be optimized relative to the pressure drop across the entire chip array. In some embodiments, optimizing the flow resistance across a single chip relative to the flow resistance across the entire chip array can impact the pressure drop across a single chip relative to the pressure drop across the entire chip array.

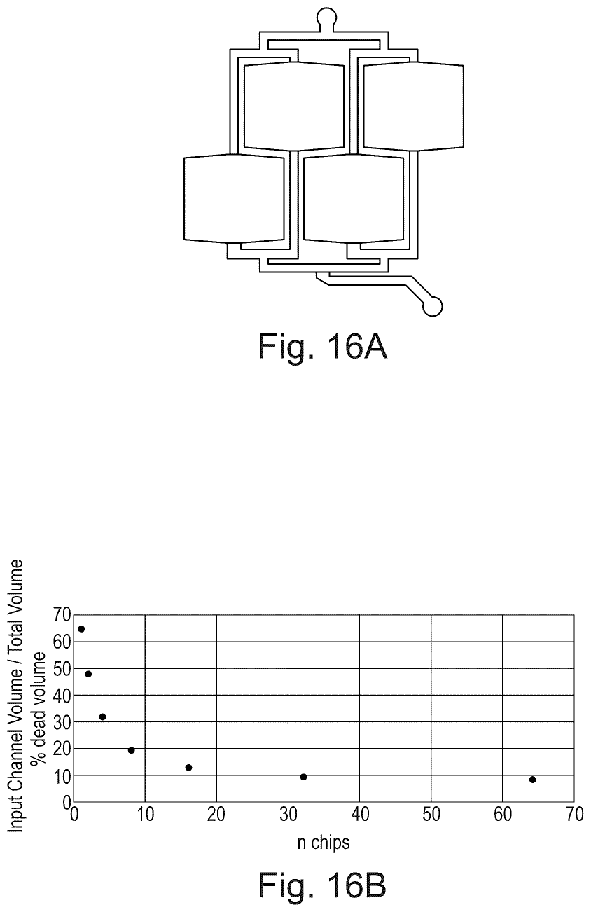

FIG. 16A illustrates an exemplary compartment comprising a 4-chip array as described elsewhere herein. FIG. 16B illustrates a plot of input channel volume/total volume as a function of chip number. The input channel volume/total volume can be inversely related to the chip number. The input channel volume/total volume can decrease as the number of chips increases. In one example, the input channel volume/total volume for a 4-chip array can range from about 30% to 40%. As more chips are added, less of the total volume can be associated with the input channels.

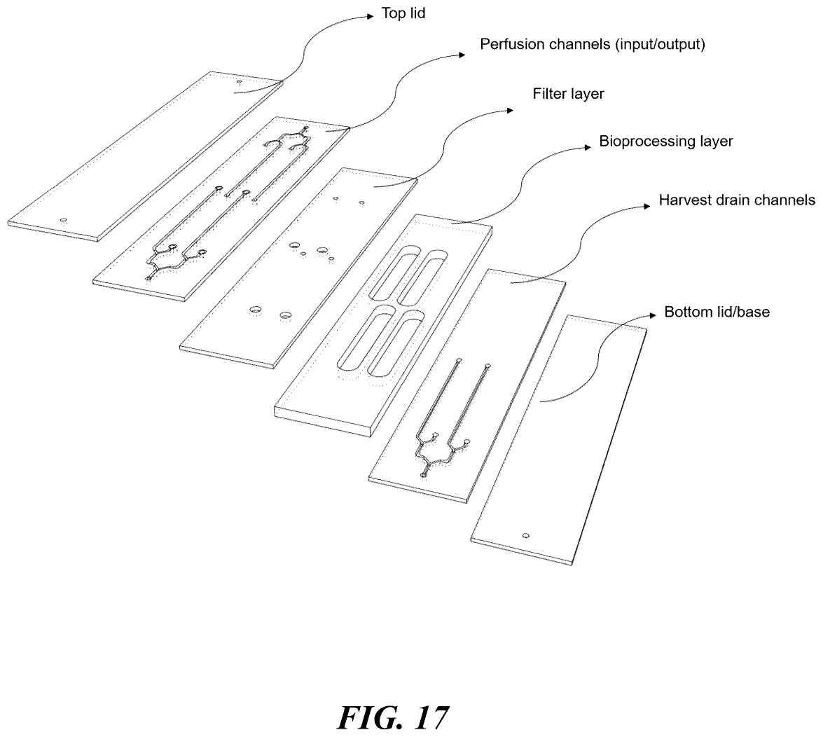

FIG. 17 illustrates a parallel array of chip in a 2×2 array. The chip array (cassette) can comprise a harvest channel layer, a drain layer, a filter layer comprising one or more filters, a feeding layer, a bioprocessing layer, and a perfusion output layer. The chip array with the various layers shown in FIG. 17 can be integrated together. In some cases, the integrated assembly can be hermetically sealed.

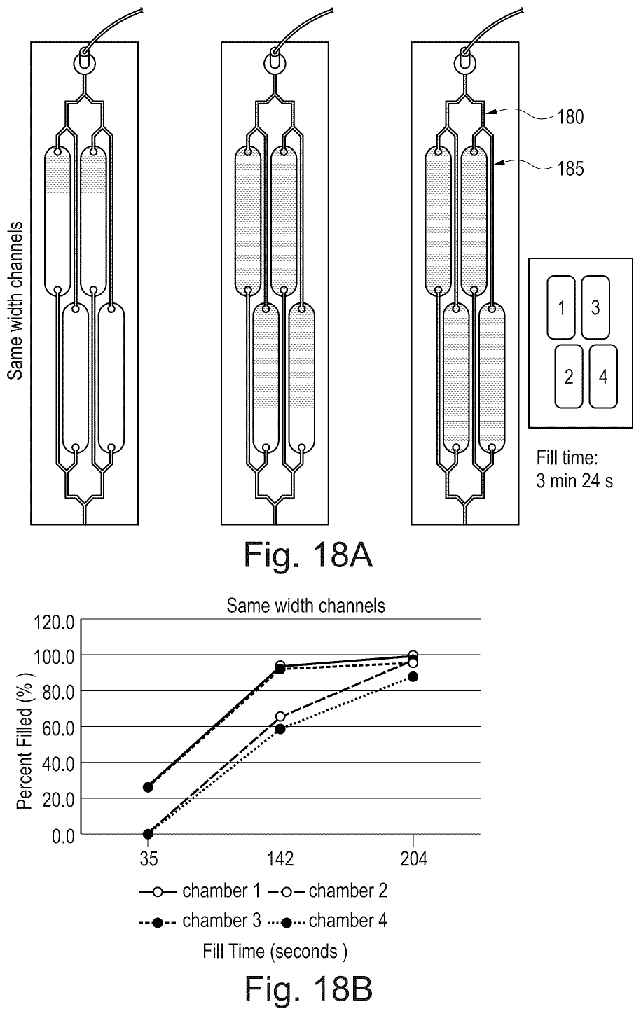

FIG. 18A illustrates the fill scheme of a cassette with a parallel array of a chip in a 2×2 array. In the cassette of FIG. 18A, the upstream portions of the channels 180 are a uniform width as the downstream channels 185. FIG. 18B shows the percent filled of the chambers of the cassette of FIG. 18A as a function of time. Data points were collected when a chamber reached 100% filled. As shown in FIG. 18B, chambers 1 and 3 were 100% filled after 142 seconds. Chambers 2 and 4 filled at a slower rate and reached 100% filled after around 204 seconds.

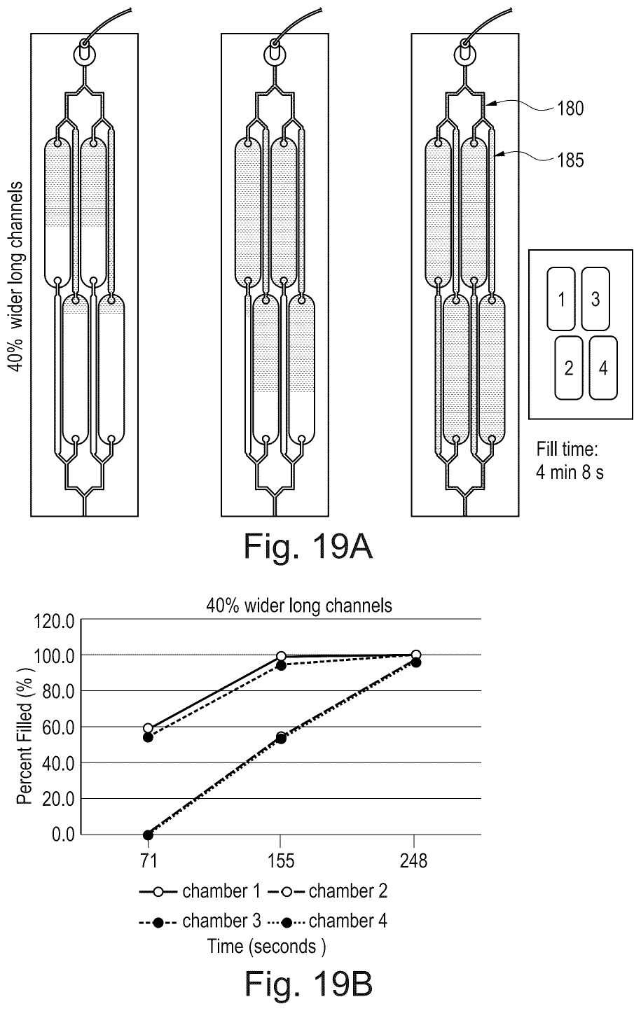

FIG. 19A illustrates the fill scheme of a cassette with a parallel array of a chip in a 2×2 array. In the cassette of FIG. 19A, the downstream longer portion of the channels 185 are 40% wider than the upstream shorter portions of the channels 180. FIG. 19B shows the percent filled of the chambers of the cassette of FIG. 19A as a function of time. Data points were collected when a chamber reached 100% filled. As shown in FIG. 19B, chambers 1 and 3 were 100% filled after 155 seconds. Chambers 2 and 4 filled at a slower rate and reached 100% filled after around 248 seconds.

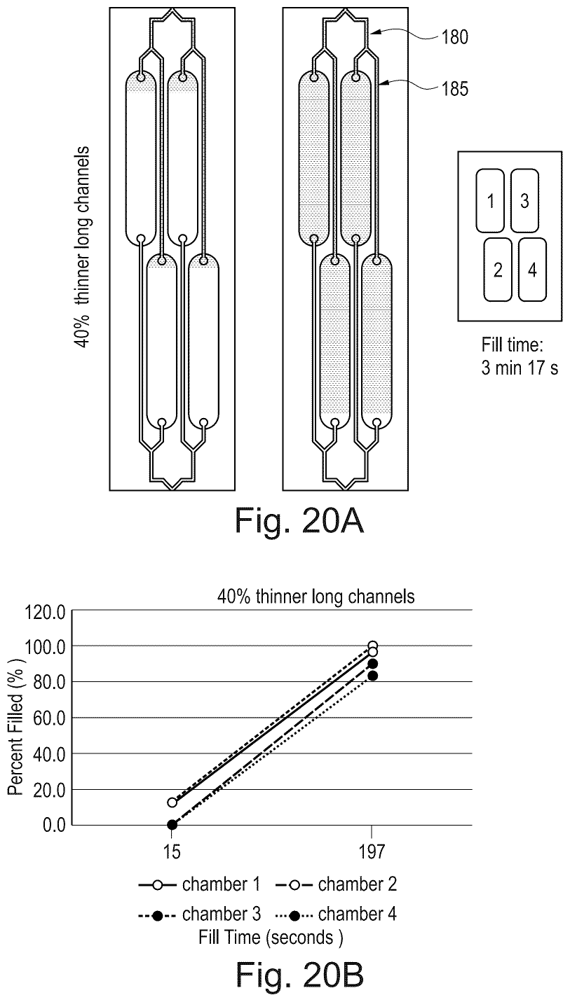

FIG. 20A illustrates the fill scheme of a cassette with a parallel array of a chip in a 2×2 array. In the cassette of FIG. 20A, the downstream longer portion of the channels 185 are 40% thinner than the upstream shorter portions of the channels 180. FIG. 20B shows the percent filled of the chambers of the cassette of FIG. 20A as a function of time. Data points were collected when a chamber reached 100% filled. As shown in FIG. 20B, chambers 1-4 filled at roughly the same rate (represented by a straight line). All chambers were 100% filled after 197 seconds. The even fill time represented in FIG. 20B is an ideal fill scheme.

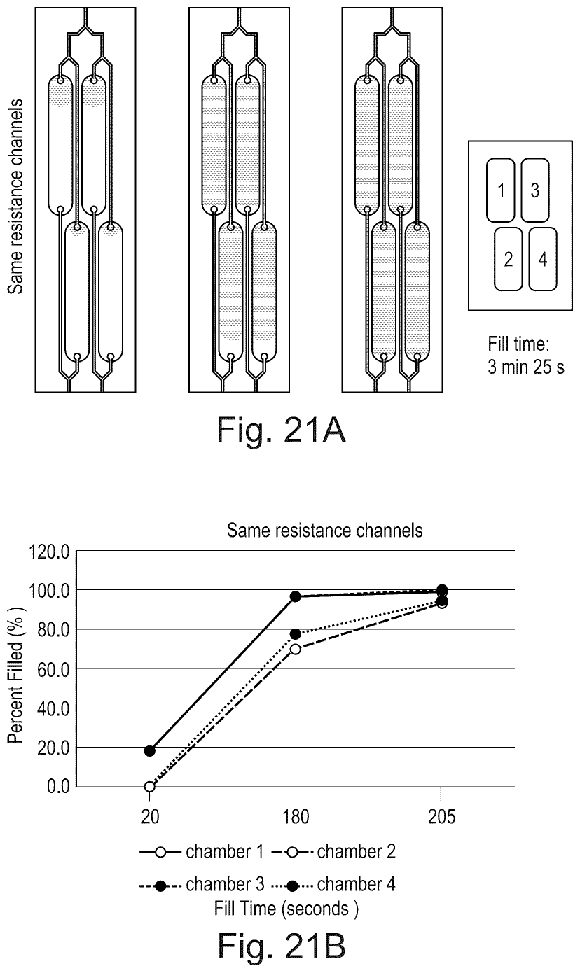

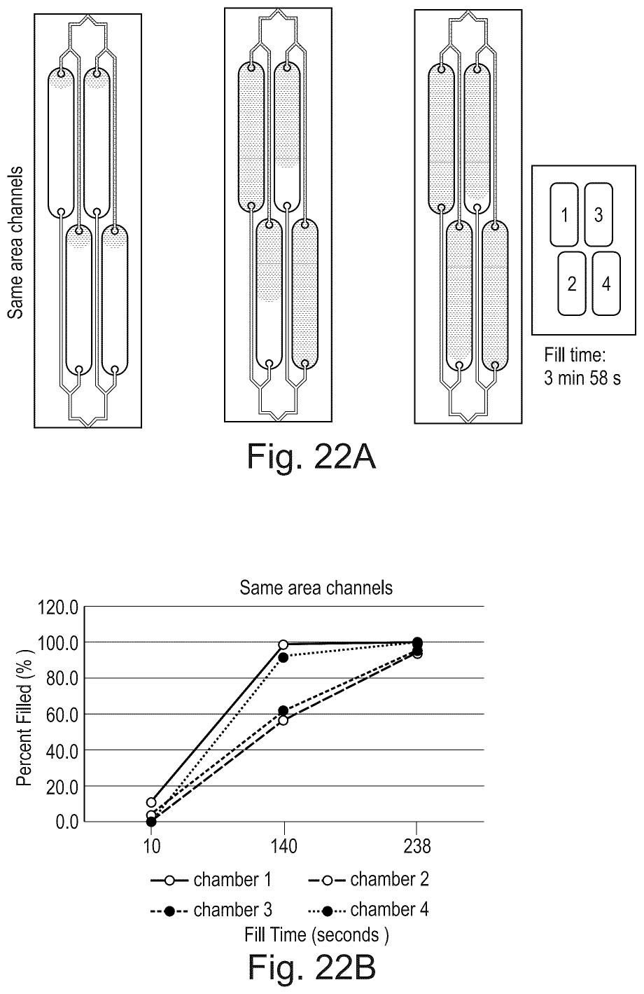

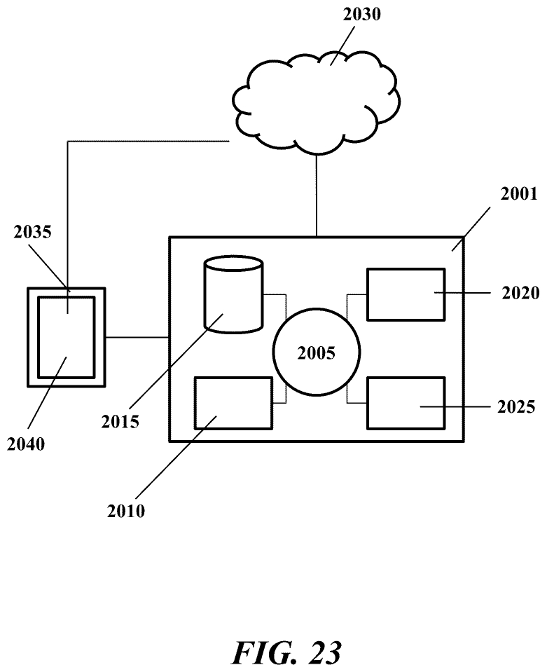

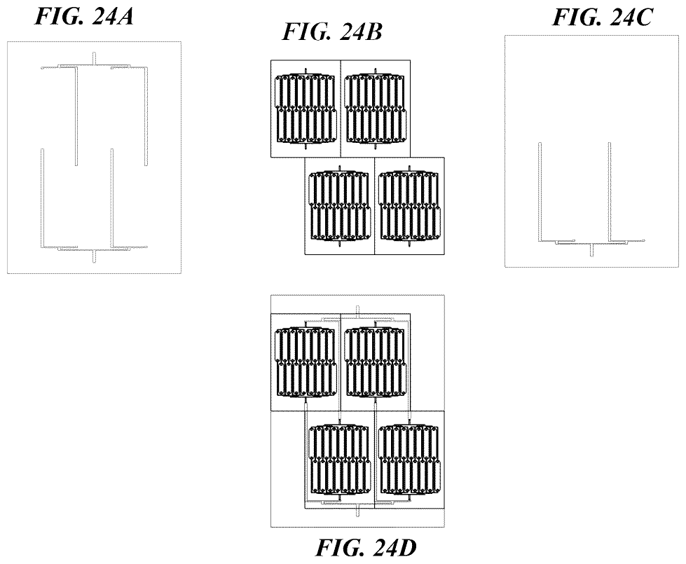

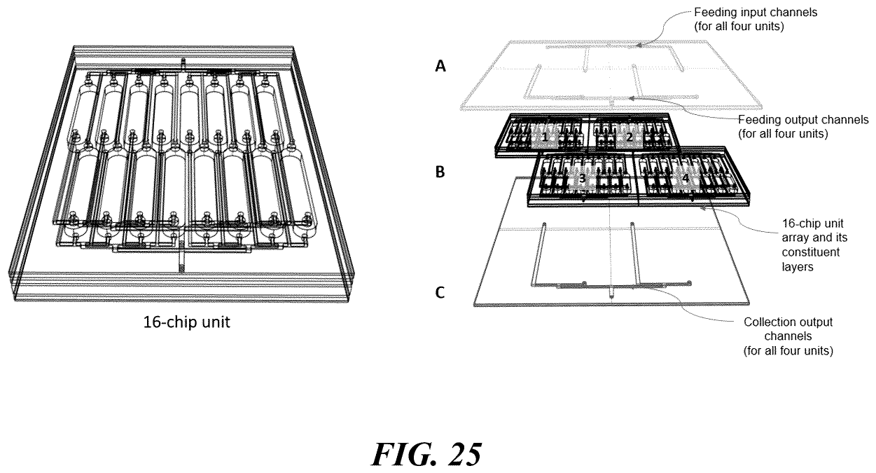

FIG. 21A illustrates the fill scheme of a cassette with a parallel array of a chip in a 2×2 array. In the cassette of FIG. 21A, all channels have the same resistance. In order for all channels to have the same resistance, channels may be sized so that, taking into account the flow being split from the upstream channels into the downstream channels, the hydraulic resistance remains constant across all channels. Hydraulic resistance can be defined as the pressure drop over the length of the channel divided by the flow rate of fluid in the channel. This can be achieved using numerical simulations or theoretical calculations. FIG. 21B shows the percent filled of the chambers of the cassette of FIG. 21A as a function of time. Data points were collected when a chamber reached 100% filled. As shown in FIG. 22B, chambers 1 and 3 were both roughly 100% filled after 180 seconds. Chambers 2 and 4 filled at a slower rate and reached 100% filled after around 205 seconds.

FIG. 22A illustrates the fill scheme of a cassette with a parallel array of a chip in a 2×2 array. In the cassette of FIG. 22A, all channels have the same cross-sectional area. FIG. 22B shows the percent filled of the chambers of the cassette of FIG. 22A as a function of time. Data points were collected when a chamber reached 100% filled. As shown in FIG. 22B, chambers 1 and 4 were both roughly 100% filled after 140 seconds. Chambers 2 and 3 filled at a slower rate and reached 100% filled after around 238 seconds.

FIGS. 24A-24D illustrates a 64-chip array based on four 16-chip units. FIG. 24A shows a perfusion inlet and filter outlet layer connecting the perfusion inlets and filter outlets of four 16-chip units. This is at the top of the 16-chip units. FIG. 24B shows a 64-chip unit layer comprising the four 16-chip units. FIG. 24C shows a harvest layer connecting the four harvest lines of the 16-chip units. This is at the bottom of the 16-chip units. FIG. 24D shows an overlay of the layers shown in FIGS. 24A-24C.

FIG. 25 illustrates an exploded view of a 64-chip array based on two 16-chip units. The chip array can comprise a layer (A) comprising feeding input channels for all four 16-chip units and feeding output channels for all four 16-chip units. The 64-chip array can comprise a layer (B) comprising the four 16-chip units. The 64-chip array can comprise a layer (C) comprising collection output channels for all four 16-chip units.

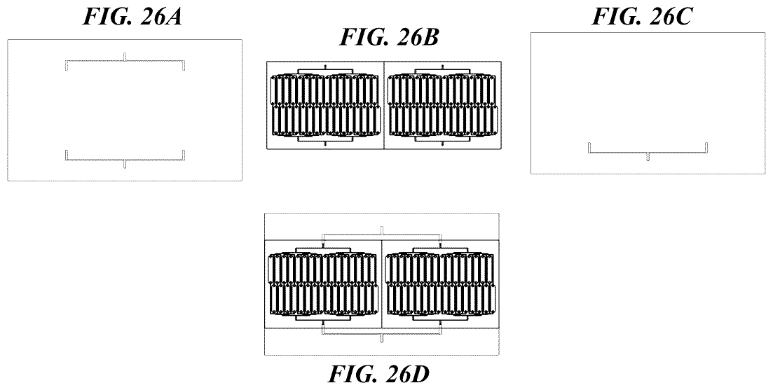

FIGS. 26A-26D illustrates a 64-chip array based on two 32-chip units. FIG. 26A shows a perfusion inlet and a filter outlet layer connecting the perfusion inlets and filter outlets of the two 32-chip units. This is at the top of the 32-chip units. FIG. 26B shows a 64-chip unit layer comprising two 32-chip units. FIG. 26C shows a harvest layer connecting the two harvest lines in the 32-chip units. This is at the bottom of the 32-chip units. FIG. 26D shows an overlay of the layers illustrated in FIGS. 26A-26C C.

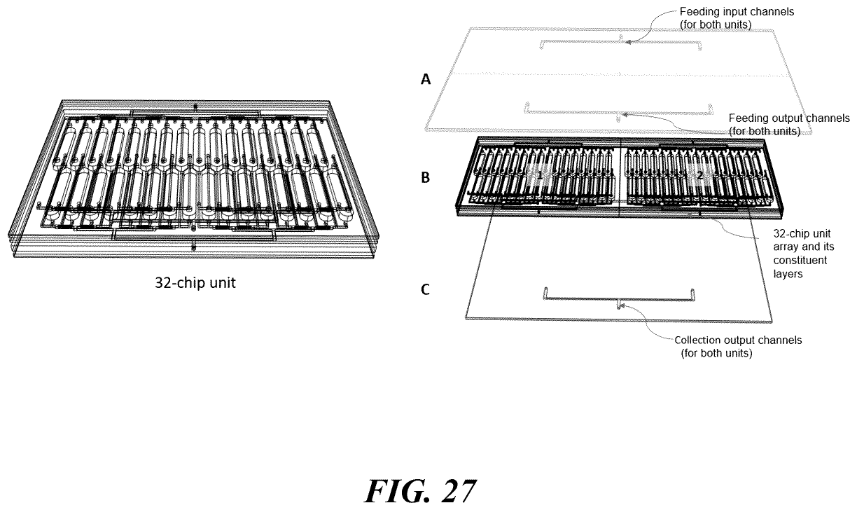

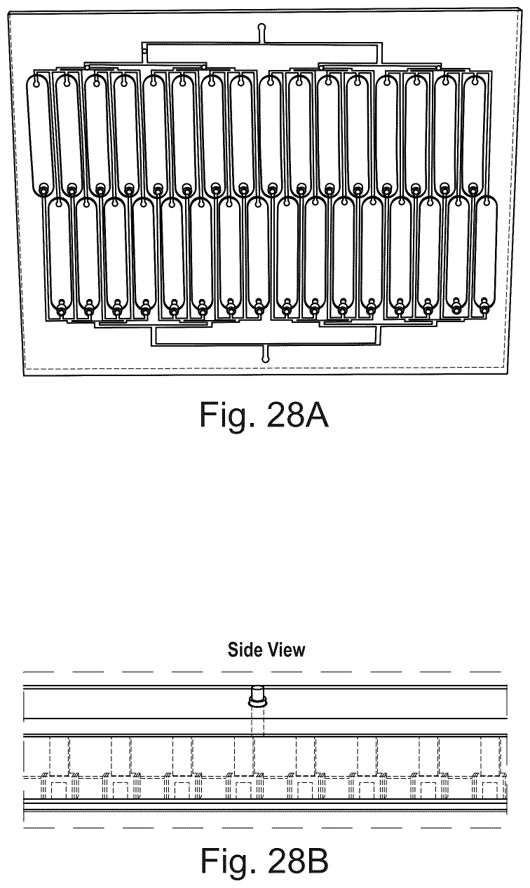

FIG. 27 illustrates an exploded view of a 64-chip array based on two 32-chip units. The chip array can comprise a layer (A) comprising feeding input channels for two 32-chip units and feeding output channels for two 32-chip units. The 64-chip array can comprise a layer (B) comprising the two 32-chip units. The 64-chip array can comprise a layer (C) comprising collection output channels for both of the two 32-chip units. FIGS. 28A and 28B respectively illustrate top and side views of an exemplary 32-chip unit.

In any of the embodiments described herein, the chip arrays may comprise input channels that feed into the bioprocessing chambers. The input channels may be configured to ensure that the cells in solution do not settle in the input channels, and instead reach the bioprocessing chambers in fluid communication with the input channels.

The total area occupied by the feeding input channels can be optimized to reduce the likelihood of cells settling in the feeding input channels. In some cases, the size, shape, and/or spatial configuration of the feeding input channels may be adjusted to minimize the amount of space (e.g., area or volume) occupied by the channels, thereby reducing the amount of dead space or dead volume available for the settling of cells. Minimizing the footprint of the channels may facilitate the settling of cells in the bioprocessing chambers of the chip arrays. The percentage of dead volume for a chip array may be computed as a ratio between (i) the total volume of the input channels and (ii) the total volume of the input channels plus the total bioprocessing volume associated with the chips in the chip array. In some cases, the percentage of dead volume for a chip array may be computed as a ratio between (i) the total volume of the input channels and (ii) the total volume of the input channels plus the total bioprocessing volume associated with the chips in the chip array plus the volume of the outlet and/or collection channels.

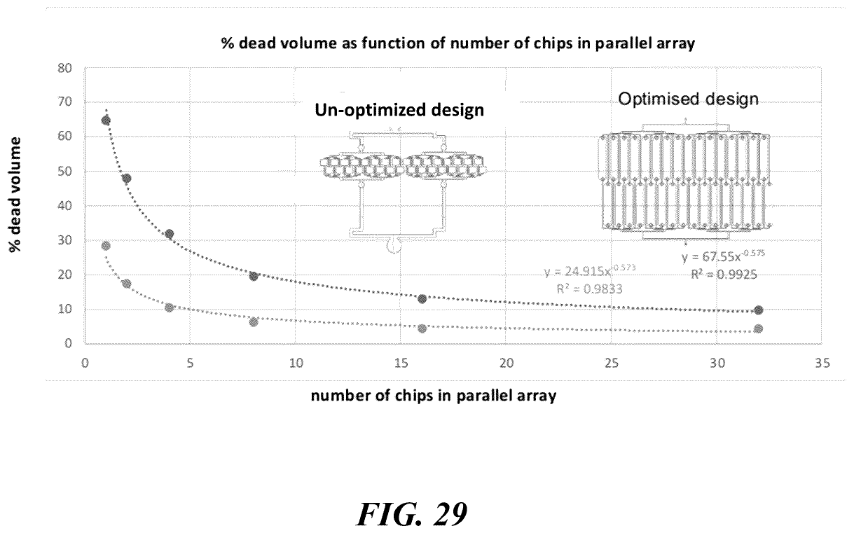

FIG. 29 illustrates a plot of a percentage of dead volume for a microfluidic device comprising a parallelized chip array as a function of a number of chips in the parallelized chip array. The percentage of dead volume in a chip array may decrease as the number of chips in the array increases. The optimized chip array designs disclosed herein may have less dead space for cells to settle compared to other existing un-optimized chip designs, and can increase the number of cells reaching the bioprocessing chamber as opposed to other portions or sections of the chips (e.g., the feeding input channels).

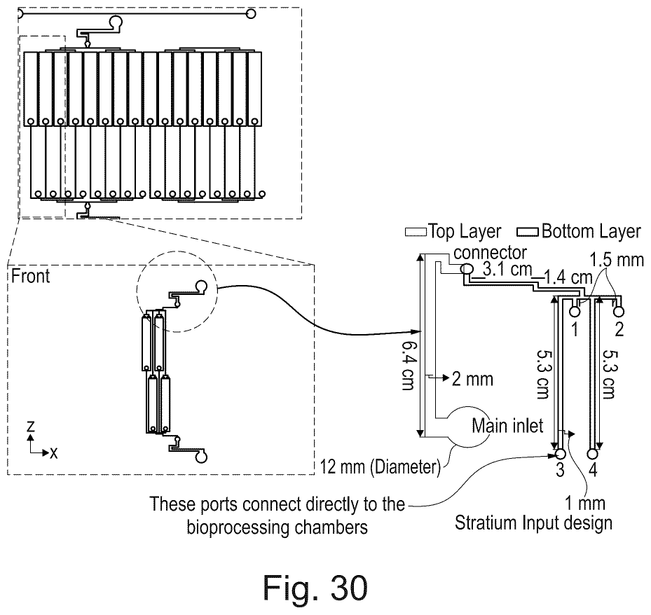

FIG. 30 illustrates a portion of a parallelized chip array. The portion may comprise one or more units of a chip array. Each unit of the chip array may comprise one or more ports that connect directly to the bioprocessing chambers of the individual chips. Each unit may comprise a multi-layer design, with a main input channel forming the top layer and one or more additional ports or channels formed on the bottom layer. Each unit may comprise a main inlet and a plurality of outlet ports. The top layer and the bottom layer may be fluidically connected via a connector.

The chip array shown in FIG. 30 can permit flushing of cells through the feeding input channels completely. In some cases, the chip array can be flushed with an anti-adherent to ensure that no cells adhere to the surfaces of the chip array channels. The anti-adherent may comprise, for example, a starch or cellulose. The chip array can then be washed with a PBS buffer, and a cell culture can be passed through the chip array using a pump.

In one instance, a 0.5 mL volume of cells was collected at each port. The above procedure was repeated and the chip array was further flushed with cell media, and another 0.5 mL of volume was collected at each port. The chip array was flushed again with cell media and another 0.5 mL of volume was collected. The cells from every port were counted to determine the total number of cells passing through the chip array and the total number of cells collected.

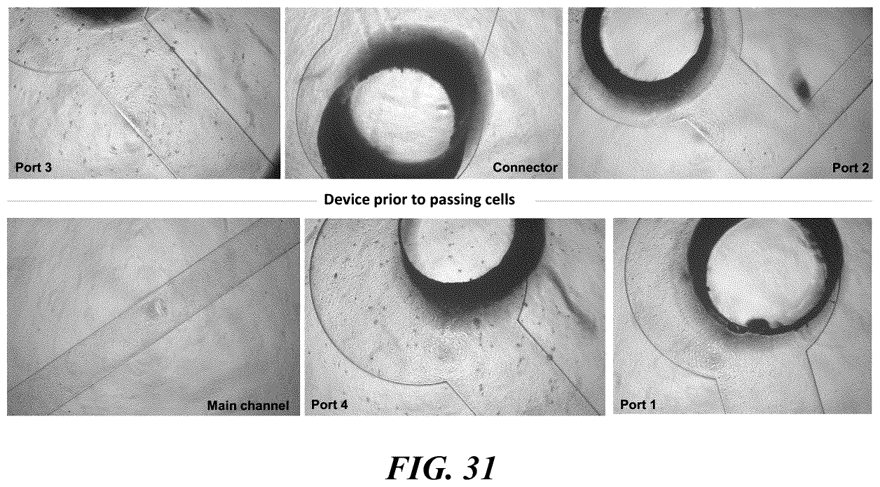

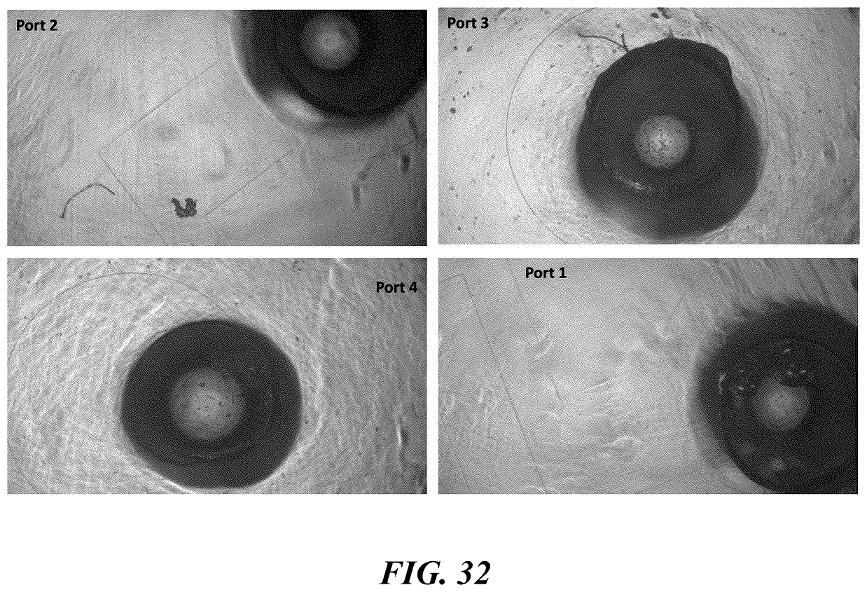

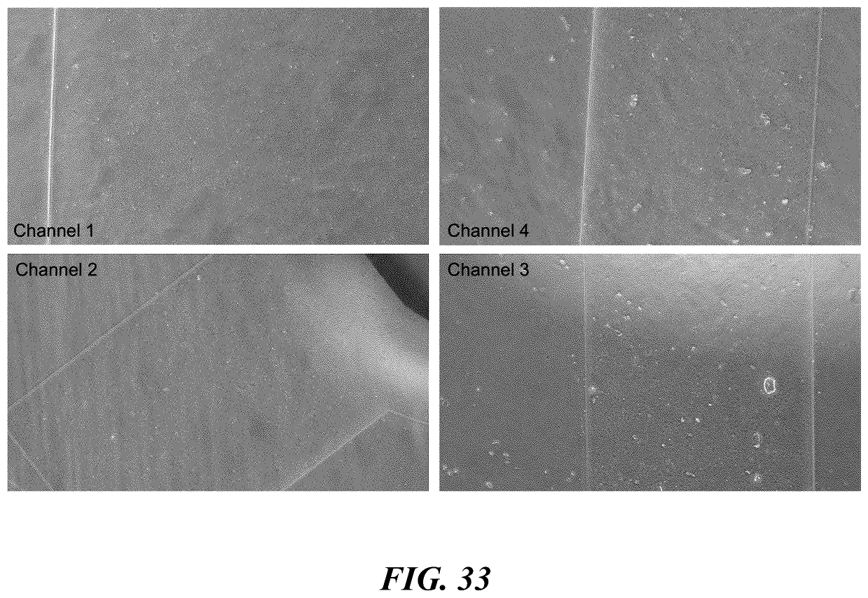

FIG. 31 illustrates various ports and channels of the chip array prior to passing cells through the chip array. FIG. 32 shows that after flushing the device, there are nearly no cells present in the outlet holes or ports, which indicates that the cells have left the input channels. FIG. 33 shows that after flushing the device, there are nearly no cells present in the channels, which also indicates that the cells have left the channels and have not settled in the feeding input channels after flushing.

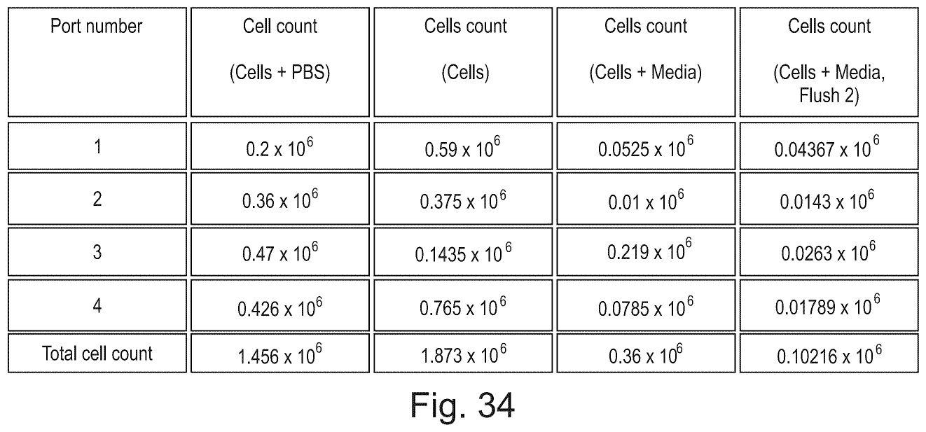

FIG. 34 illustrates a table showing the cell counts for the cells collected from a microfluidic device comprising an array of chips after the channels of the chip array are flushed. The cells used may comprise Jurkat cells. The initial volume of cells was 4 mL (kept in a 15 mL falcon tube) and the initial seeding concentration of cells was 0.962×10⁶ cells per mL. The total number of cells passed through the chip array was about 3.848×10⁶ cells. The total number of cells collected through all of the ports and channels of the device after flushing was about 3.792×10⁶ cells, which corresponds to a cell seeding efficiency of over 98% and only a 2% loss.

The results shown in FIG. 34 were compiled over multiple runs. In one run, a plurality of cells and a balanced salt solution (phosphate buffered saline or PBS) were passed through the chip array with 0.5 mL collected at each port. In another run, cells were passed through the chip array with 0.5 mL collected at each port. In another run, cell media was passed through the chip array with 0.5 mL collected at each port. In another run, the chip array was flushed again with the cell media, with 0.5 mL collected at each port. After the second flushing of the chip array with the cell media, the volume left in the falcon tube was around 250 μL, the final concentration of cells was about 0.90×10⁶ cells per mL, and the total number of cells was about 0.22×10⁶ cells. The images and results shown in FIGS. 31-34 indicate that there were no cells visible in the ports or channels after flushing, and that the vast majority of cells do not settle in the feeding input channels after flushing the chip array with cell media.

### Detachable Filter

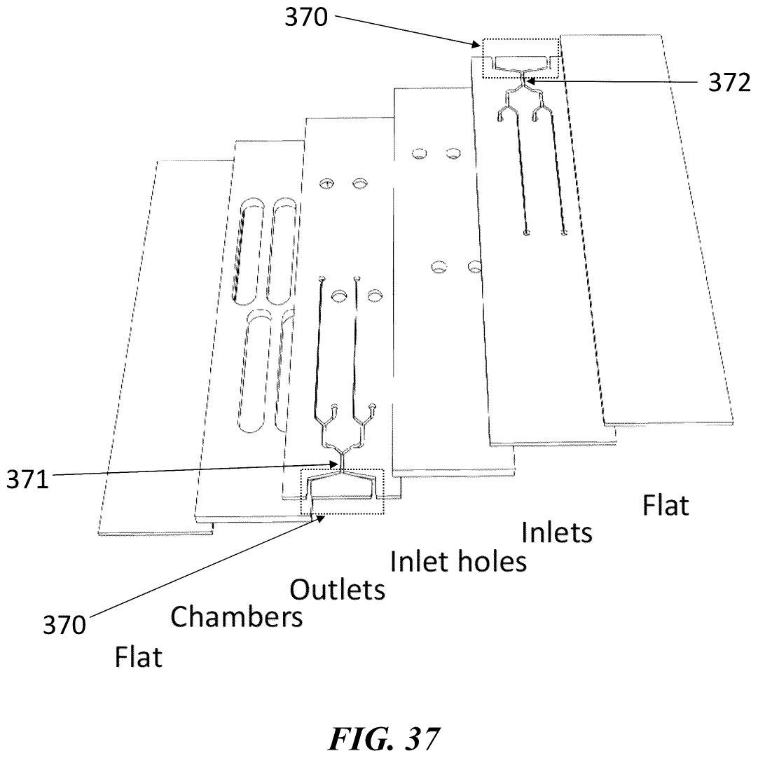

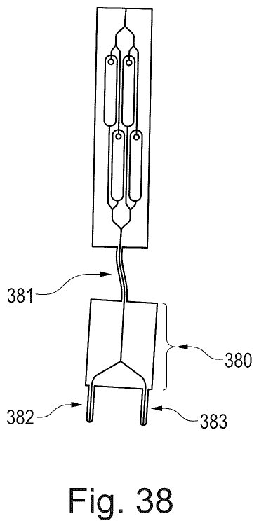

In some cases, a detachable filter unit can be added to the input or output channel of any of the cassettes described herein. As shown in FIG. 37, a detachable filter 370 can connect to an outlet port 371 or an input port 372. In some cases, the detachable filter comprises a split point that divides the output 371 of a cassette into two or more output streams or combines two or more input streams into one input channel 372 that will be fed to a cassette. In some cases, the detachable filter can be used to control flow to/from an input or output. For example, as shown in FIG. 38, a detachable filter 380 can be attached to an outlet channel 381 of a cassette. The detachable filter 380 can divide the outlet channel 381 into one or more additional output streams, 382 and 383. One of the output streams can be blocked so that fluid only flows through one of the additional output streams, either 382 or 383. Fluid can be pulled via one or both of the output streams 382 and 383 via a syringe pump or a suction generated by a negative pressure source. In some cases, the output streams 382 and 383 utilize different filters or filtering methods. As such, a user can select which filtering method to use by blocking fluid flow through the non-desired output channel.

### Fused Bioprocessing Chamber

In some embodiments, bioprocessing chambers can be connected or “fused”, while still maintaining the same number of bifurcations as other chip configurations with bioprocessing chambers that have not been connected or “fused.” These fused bioprocessing chambers can further facilitate communication between cells in a much larger space without having to separately seed multiple chambers. This is because in a classic 2×2 array, the cell numbers in each of the four chambers are not significantly different, while in the fused array, only one or two chambers can have approximately equal numbers (instead of all four chamber). The same level of bifurcation can be maintained from the primary input to the bioprocessing chambers since this bifurcation can optimize fluid distribution while minimizing fluid pressure drops. In some cases, an advantage of a fused chamber is that it is not necessary to rely on the exact same number of cells reaching each chamber (when seeding) and the exact same flow rate in each chamber (e.g., when perfusing). If there is any imbalance in one of the channels (e.g., because of imperfect fabrication, bubble, debris), the fact that the chambers are fused can compensate for this through agitation of the fluid in the fused chamber, therefore homogenizing the cells and the fluid across the fused chamber.

In some cases, a cassette comprises (a) a branched network of feeding input channels in a first plane, (b) a bioprocessing layer in a second plane comprising a fused bioprocessing chamber fluidically connected to the branched network of feeding input channels in the first plane; and (c) a branched network of feeding output channels fluidically connected to the fused bioprocessing chamber. In some cases, the cassette permits a fluid to flow from the branched network of feeding input channels in the first plane, through the fused bioprocessing chamber in the second plane, and through the branched network of feeding output channels. The fused bioprocessing chamber may have one or more sub-chambers that are separated by a liquid permeable membrane. The liquid permeable membrane may be impermeable to cells or other biological samples. In some cases, the input and output channels are located in the same plane. This plane may be located above the bioprocessing chamber.

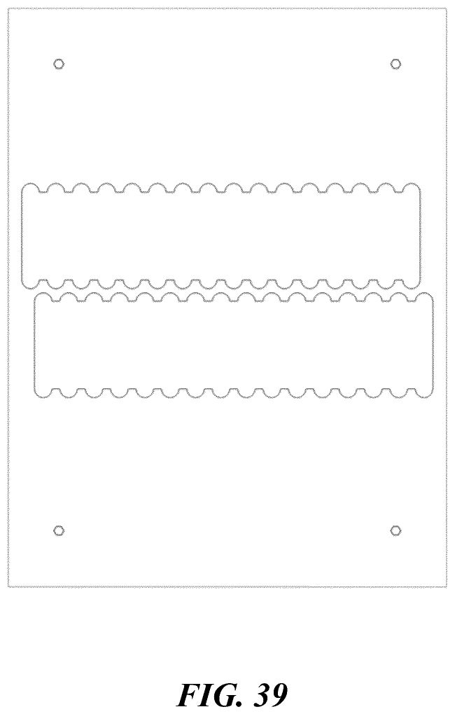

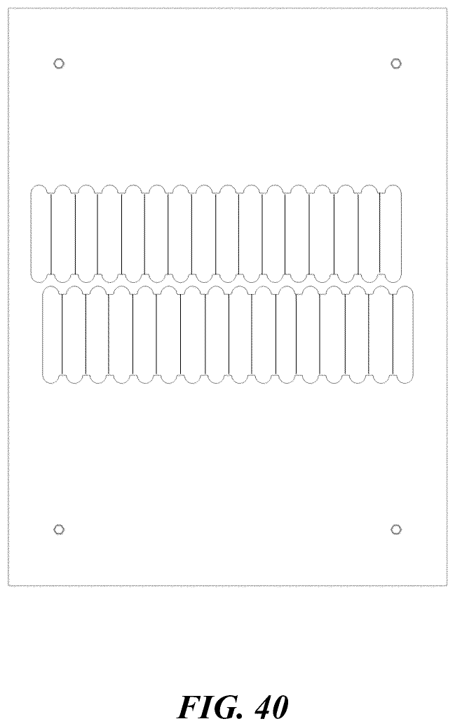

FIG. 39 schematically illustrates a 32-chip cassette with two fused chambers, in accordance with some embodiments. Rather than having 32 individual chambers, the chip can have two fused chambers. Each fused chamber can correspond to 16 individual chambers that have been fused together. In some cases, both chambers have approximately equal numbers of cells.

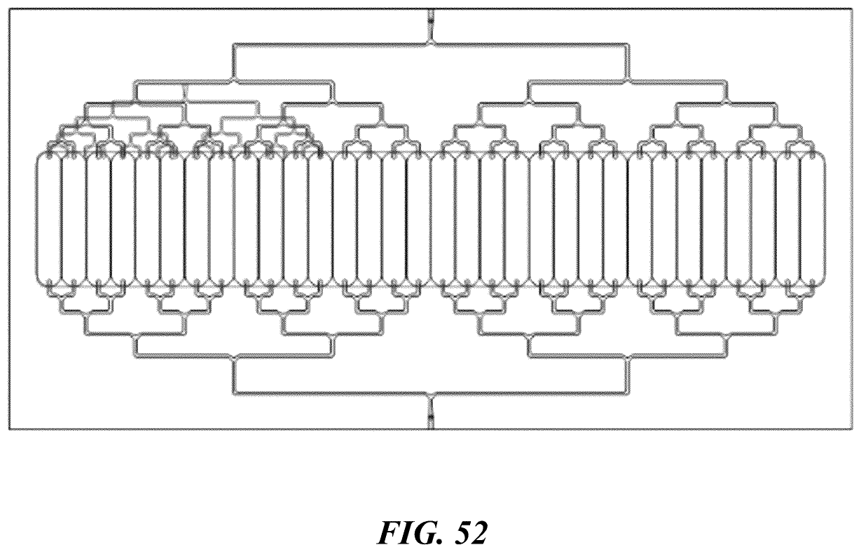

FIG. 40 schematically illustrates a 32-chip cassette with chambers separated by liquid permeable walls, in accordance with some embodiments. Rather than having 32 individual chambers, a chip may have two fused chambers. Each fused chamber may correspond to 16 individual sections that are each separated by a liquid permeable wall. This can allow for fluidic communication within the entire chamber while maintaining features of the chip. In some cases, the liquid permeable wall is impermeable to cells. In some cases, both chambers have approximately equal numbers of cells. Within each fused chamber, cells can be evenly distributed between the 16 individual sections. FIG. 52 schematically illustrates a 32-chip cassette with a single chamber divided into 32 sections by liquid permeable walls.

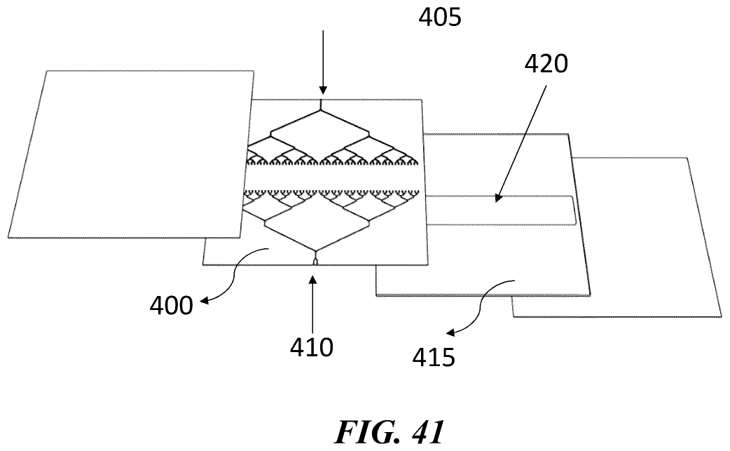

FIG. 41 schematically illustrates an example of a 64-chip one row cassette with one fused bioprocessing chamber, in accordance with some embodiments. In some cases, a first layer 400 can comprise one or more feeding output channels 405 and one or more feeding input channels 410. In some cases, all of the feeding input channels are located on the same plane as each other. In some cases, all of the feeding output channels are located on the same plane as each other. The one or more feeding output channels 405 and the one or more feeding input channels 410 may be located on the same plane. A second layer 415 may comprise one fused bioprocessing chamber 420. The second layer 415 may be on a separate plane than the feeding input and output channels.

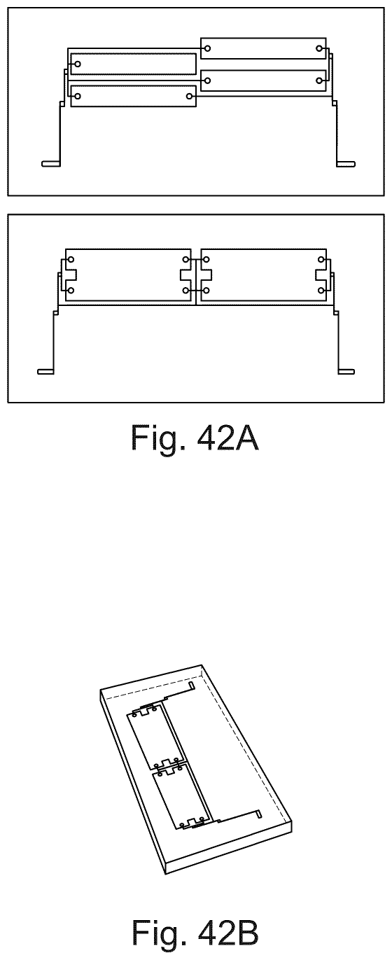

FIG. 42A and 42B schematically illustrate an example of a cassette with parallel array of chips in which the chips are fused, in accordance with some embodiments.

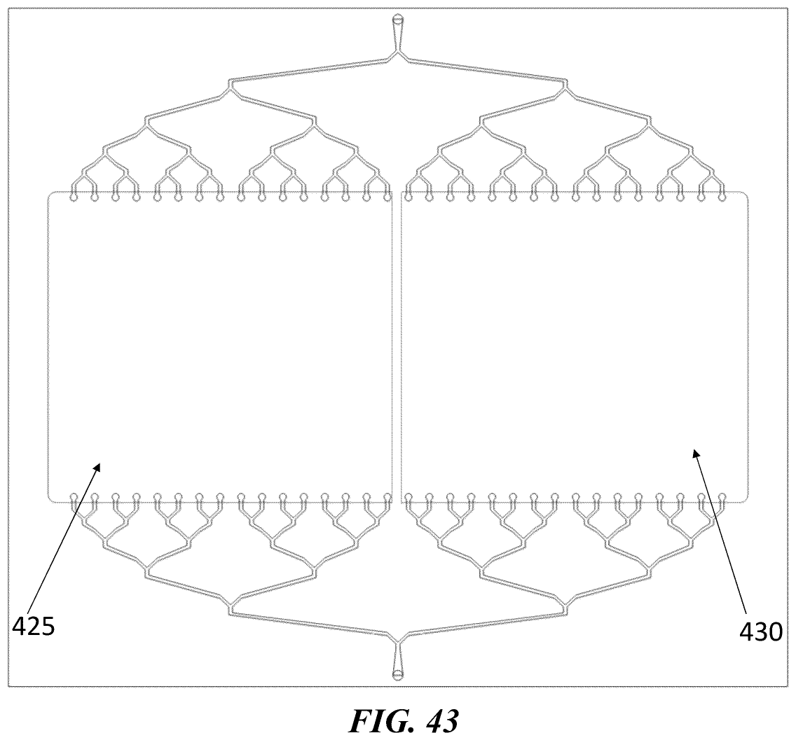

FIG. 43 schematically illustrates an example of a 32-chip cassette with fused chambers divided into two compartments down the middle, in accordance with some embodiments. The same level of bifurcation can be maintained as a standard 32-chip cassette. Half of the input channels can feed to a first fused chamber 425 and half of the input channels can feed to a second fused chamber 430. In some cases, the cell count in chambers 425 and 430 are roughly equivalent.

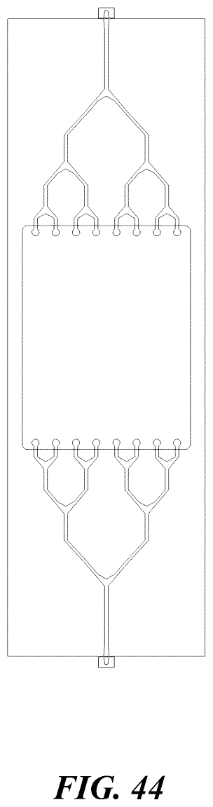

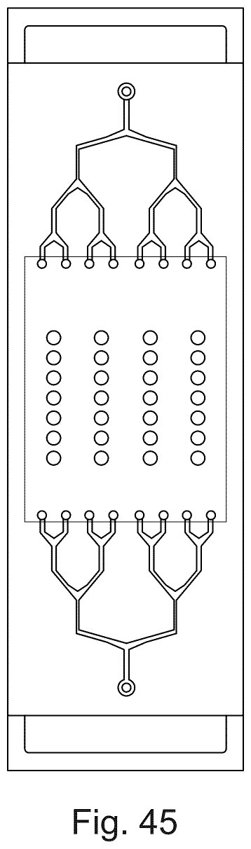

FIG. 44 schematically illustrates an example of an 8-chip bifurcated cassette with a single bioprocessing chamber, in accordance with some embodiments. The same level of bifurcation can be maintained as a standard 8-chip cassette. In some cases, a gas lid layer may be added on top of a channel layer. FIG. 45 schematically illustrates an example of an 8-chip bifurcated cassette with a single chamber and a gas lid layer, in accordance with some embodiments. In some cases, the gas lid layer comprises one or more holes that allow gas to enter into a bioprocessing chamber. The gas lid layer may be attached to a permeable membrane that allows gas to diffuse through. In some cases, the permeable membrane is flexible and thin. The gas lid layer can be an additional rigid layer that provides structural strength to the permeable membrane, e.g, prevents bulging of the membrane when the pressure inside the bioprocessing chamber becomes higher than that of the environment in which the bioreactor sits. The material used for the gas lid layer can be impermeable to gas, so the holes are there to allow the gas to pass through the permeable membrane. In some cases, the gas permeable membrane is located directly below the gas lid layer.

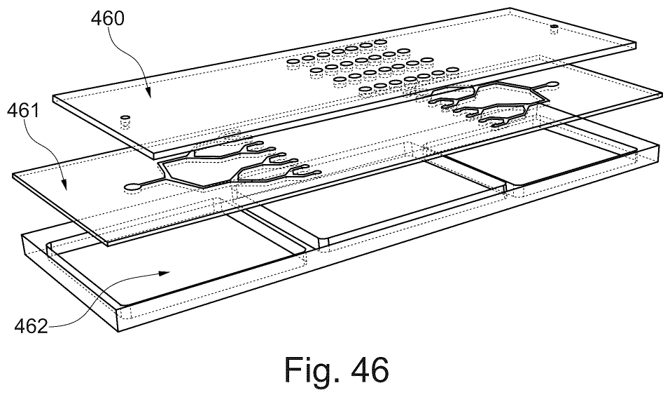

FIG. 46 schematically illustrates an example of an 8-chip bifurcated cassette with a gas lid layer 460, a channel layer 461 comprising bifurcated channels, and a bioprocessing layer 462 comprising a single fused bioprocessing chamber.

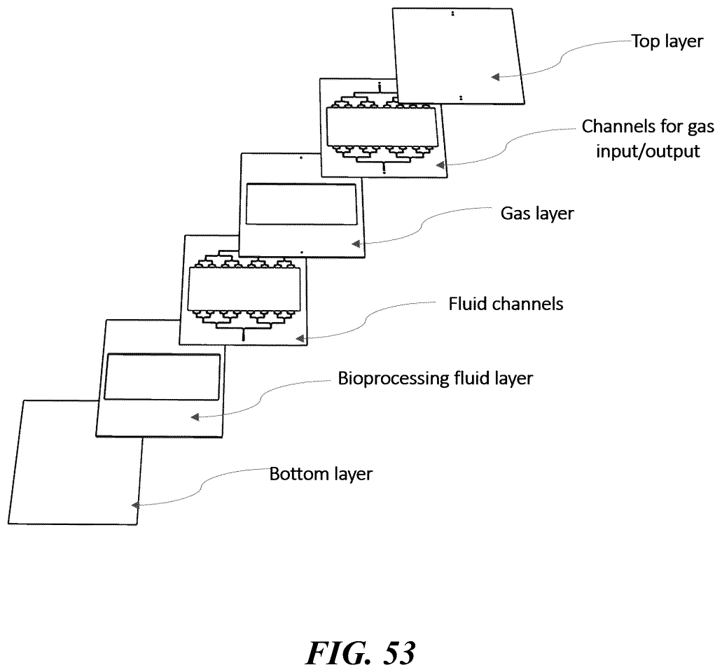

FIG. 53 schematically illustrates a cassette with a gas layer and gas input and outlet channels. In some cases, the cassette permits a gas to flow from a branched network of gas input channels, through a gas layer, and through a branched network of gas output channels in the third plane. In some cases, a cassette comprises a bioprocessing layer and a gas layer. The gas layer may be located on top of the bioprocessing layer. Once gas has been fed into the gas layer, it may occupy the overhead space above the bioprocessing layer or one or more bioprocessing chambers. In some cases, the bioprocessing layer and gas layer are not separated by a membrane, thus allowing gas to dissolve directly into the bioprocessing fluid. In some cases, a branched network of gas input channels feeds the gas layer. The branched network of gas input channels may all be in a single plane. In some cases, the branched network of gas input channels are located above the gas layer. Gas may exit the gas layer through a branched network of gas output channels. The gas output channels may all be located in a single plane. In some cases, the gas output channels are located above the gas layer. In some cases, the gas input channels and gas output channels are located in the same plane. The cassette may be stacked such that the gas layer and gas input/output channels are above the fluid input/output channels and above a bioprocessing layer or bioprocessing chamber. As shown in FIG. 53, the cassette can have an additional top layer above the gas layer. The cassette can have an additional bottom layer located below the bioprocessing layer. In some cases, the order of layers in a cassette (listed from top to bottom) is as follows: a top layer or ceiling, gas input and output channels, gas layer, bioprocessing fluid input and output channels, bioprocessing layer, and bottom layer.

### Fluid Channel Designs

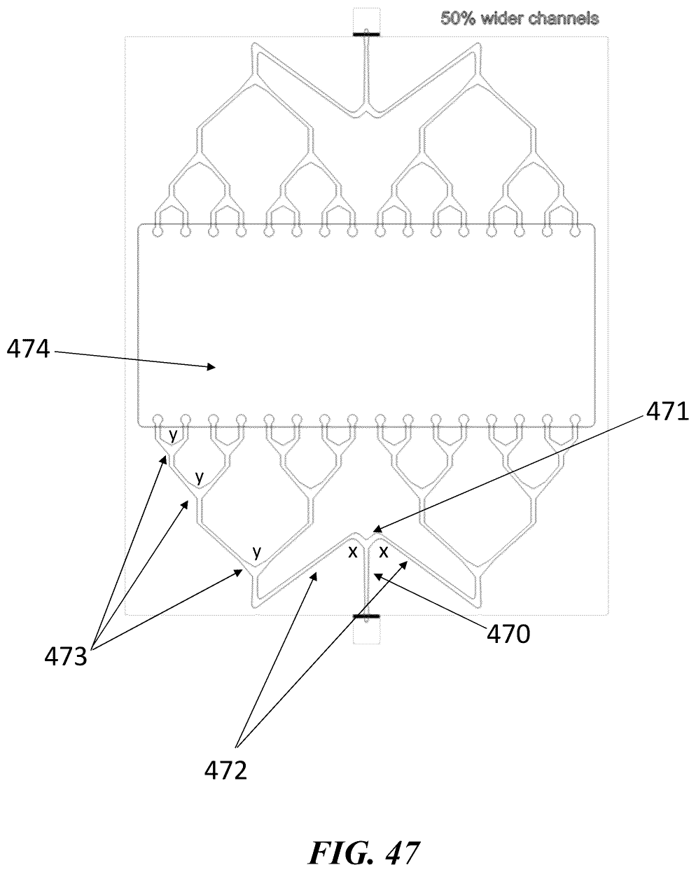

Fluid channels may be designed and optimized to achieve certain characteristics, such as uniform seeding or injection rates. FIG. 47 schematically illustrates an example of a bifurcated cassette with a single bioprocessing chamber. In some cases, the width of the channels may be between 1.5 mm and 1.8 mm in width. In some cases, widening the channels of a cassette results in a lower cassette footprint, saving space and reducing cost of materials. In some cases, an input channel 470 is split into two downstream channels 472 at a split point 471. The two downstream channels 472 can be angled towards the input channel 470 at an angle represented by x. In some cases, the two downstream channels 472 are angled towards the input channel 470 at an angle x of less than 90°. In some cases, the downstream channels 472 split to form additional downstream channels. Channels 472 may each split into two channels at a split point 473. In some cases, the two downstream channels split from the upstream channel at an angle represented by y. In some cases, y is less than or equal to 180°. These additional downstream channels may continue to split into two or more channels via additional split points 473 until all channels reach a bioprocessing chamber 474.

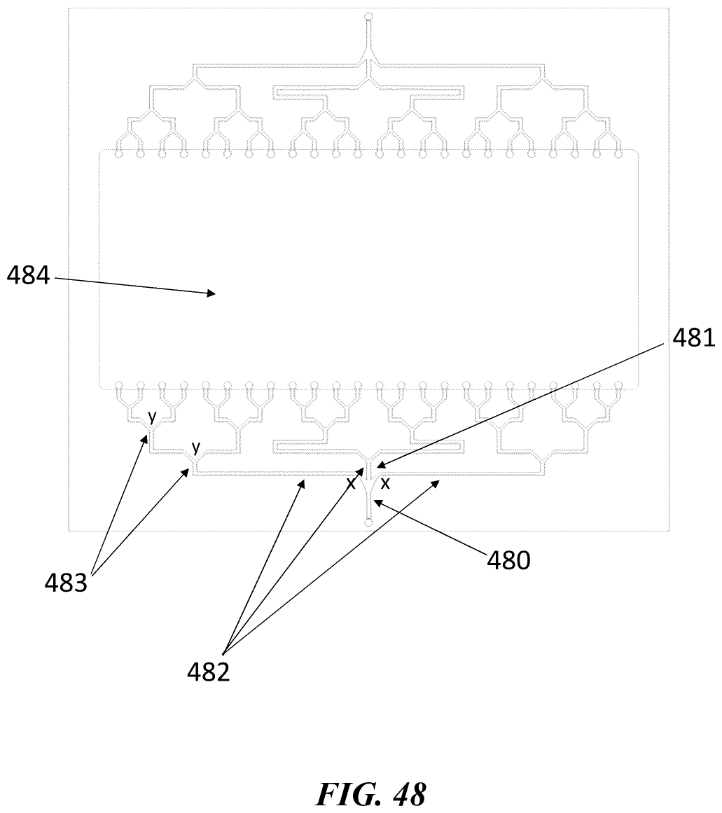

FIG. 48 schematically illustrates an example of a cassette with a 1-to-3 split followed by bifurcated splits and a single chamber, in accordance with some embodiments. In some cases, an input channel 480 is split into three downstream channels 482 at a split point 481. Two downstream channels 482 can be angled from the input channel 480 at an angle represented by x. A third downstream channel 482 is in the same direction as the input channel 480. In some cases, the two downstream channels 482 are angled from the input channel 480 at an angle of 90°. In some cases, the downstream channels 482 split to form additional downstream channels. Channels 482 may each split into two channels at a split point 483. In some cases, two downstream channels split from the upstream channel at an angle represented by y. In some cases, y is less than or equal to 180°. These additional downstream channels may continue to split into two or more channels via additional split points 483 until all channels reach a bioprocessing chamber 484.

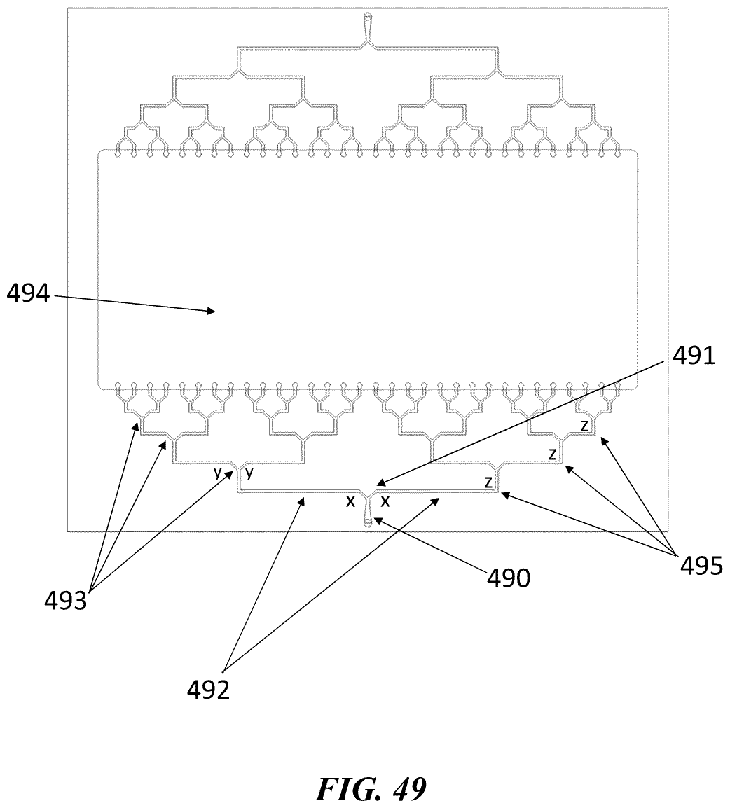

FIG. 49 schematically illustrates an example of a cassette with a 1-to-2 split followed by bifurcated splits with straight channel corners and a single chamber, in accordance with some embodiments. In some cases, an input channel 490 is split into two downstream channels 492 at a split point 491. The two downstream channels 492 can be angled from the input channel 490 at an angle represented by x. In some cases, the two downstream channels 482 are angled from the input channel 480 at an angle x of 90°. In some cases, the downstream channels 492 split to form additional downstream channels. Channels 492 may each split into two channels at a split point 493. In some cases, two downstream channels split from the upstream channel at an angle represented by y. In some cases, y is less than or equal to 180°. In some cases, y is equal to 90°. Angles x and y may be the same. These additional downstream channels may continue to split into two or more channels via additional split points 493 until all channels reach a bioprocessing chamber 494. In some cases, a channel changes direction at an angle represented by z at a channel corner 495. In some cases, channel corners 495 have an angle z of 90°.

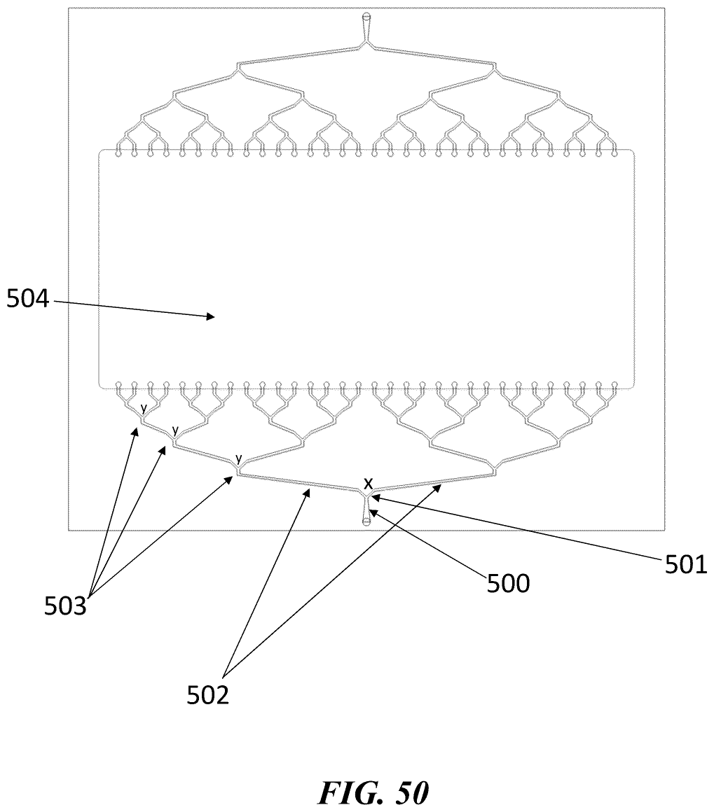

FIG. 50 schematically illustrates an example of a cassette with a 1-to-2 split followed by bifurcated splits with inclined channels and a single chamber, in accordance with some embodiments. In some cases, an input channel 500 is split into two downstream channels 502 at a split point 501. Two downstream channels 502 can be angled from the input channel 500 at an angle represented by x. In some cases, x is less than or equal to 180°. In some cases, the downstream channels 502 split to form additional downstream channels. Channels 502 may each split into two channels at a split point 503. In some cases, two downstream channels split from the upstream channel at a split point 503 at an angle represented by y. In some cases, y is less than or equal to 180°. Angles x and y may be the same. These additional downstream channels may continue to split into two or more channels via additional split points 503 until all channels reach a bioprocessing chamber 504. In some cases, the inclined channel configuration, represented by FIG. 50, reduces or eliminates bubbles within the channels.

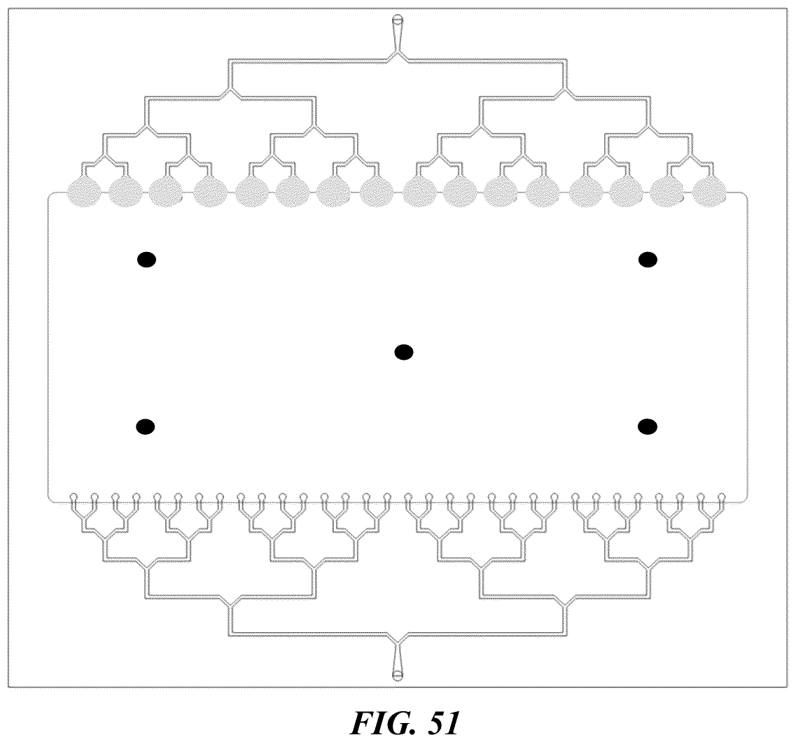

FIG. 51 schematically illustrates an example of a cassette with a 1-to-2 split followed by bifurcated splits and a single chamber, where the channel outputs from the single chamber contain a filter, and wherein the single chamber has one or more sensors, in accordance with some embodiments. One or more sensors, represented by black dots in FIG. 51, may be located throughout a bioprocessing chamber. Sensors may be used to measure pH or dissolved oxygen concentrations, for example. Sensor patches can be placed inside the bioprocessing chamber. Sensor patches can be in fluid contact with the fluid in the bioprocessing chamber. In some cases, patches are infused with a solution. The sensor may communicate with an optical fiber placed outside of the cassette. In some cases, the optical fiber emits light into or onto the sensor patch, which can reflect light back to the fiber. Characteristics of the reflected light can vary depending on the pH or dissolved oxygen concentration in the bioprocessing chamber. In some cases, two types of sensors are used (one for pH and one for dissolved oxygen). A module can be used to analyze the reflected light and provide a pH or dissolved oxygen value based on a calibration curve. Additionally, one or more filters, represented by gray dots, can help in blocking cells from prematurely exiting the bioprocessing chamber, ensuring that they stay detained inside the bioprocessing chamber.

### Applications

In some embodiments, the harvested cells from the presently disclosed chips can be used for various applications. The applications can include, for example, regenerative medicine, treatment of diabetes, cancer, and/or treatment of cardiac-related diseases or neurogenerative diseases. In some cases, the application can include autologous cell transplantation, allogenic cell transplantation, or reinfusion of cells in a patient.

The chips and cassettes disclosed herein can yield a large number of cells after cell expansion occurs. In some cases, at least about 1 million, 10 million, 100 million, 150 million, 500 million, 1 billion, 5 billion, or more cells can be harvested from the chips disclosed herein. The cells can comprise, for example, human cells (e.g., stem cells, bone cells, blood cells (e.g., white blood cells (monocytes, lymphocytes, neutrophils, eosinophils, basophils, and macrophages), red blood cells (erythrocytes), or platelets), muscle cells, fat cells, skin cells, nerve cells, immune cells (e.g., T-cells, B-cells or NK cells, lymphocytes, neutrophils, or monocytes/macrophages), cancer cells (e.g., cells associated with carcinoma, sarcoma, melanoma, lymphoma, or leukemia), or non-human cells (including, for instance, animal cells, plant cells, bacterial cells, fungal cells, etc.). Plant cells may include, for example, collenchyma, sclerenchyma, parenchyma, xylem or phloem. Bacterial cells may include, for example, spherical bacterial cells (cocci), rod-shaped bacterial cells (bacilli), spiral bacterial cells (spirilla), comma bacterial cells (Vibrios), or corkscrew bacterial cells (Spirochaetes). Fungal cells may include, for example, hyphae, yeast cells, spores, Chytridiomycota (chytrids), Zygomycota (bread molds), Ascomycota (yeasts and sac fungi), and the Basidiomycota (club fungi). In some cases, the cells may comprise chimeric antigen receptor T-cells.

### Advantages

The systems of the present disclosure can provide a multi-functional design with numerous advantages over other systems. The systems referred to herein can comprise any of the chips, chip arrays, cassettes, and other devices, hardware, or apparatuses described herein.

A device provided herein can be closed at all times, i.e., operations can be carried out in a closed environment (no opening of the chip at any time). The presently disclosed chips, chip arrays, and cassettes can be configured to carry out a plurality of functions in situ (cell seeding, perfusion, sampling, transduction, differentiation, purification, cell harvest) without opening the chip or cassette.

### Chip Composition

When cells are provided to the chips described herein, the cells can settle on or come in contact with cyclic olefin copolymer (COC), which can possess glasslike clarity that can exceed thermoplastic substitutes such as polycarbonate. COC can be sterilized using standard methods (e.g., steam, ethylene oxide, gamma irradiation, and hydrogen peroxide) without altering its properties. It can also permit UV transmission, which can be best suited for diagnostic analysis. COC can also have low leachables and extractables, making it best suited for direct drug contact. It can be classified USP Class VI and is ISO 10993 compliant including biocompatibility, USP 661.1 and FDA drug and device master files.

In some cases, the chip or cassette can comprise, for example, a polydimethylsiloxane (PDMS) component, which can form the wall of the bioprocessing chambers, as well as the top layer, which can form part of its ceiling. PDMS can have gas permeability, which can be advantageous for cell growth. PDMS can permit gas equilibration between the bioprocessing chamber and that of the surrounding controlled environment (e.g., incubator), and can withstand autoclave conditions. In some cases, the PDMS component can be replaced with another gas permeable polymer. Cells can settle on the COC portion of the bioprocessing chamber.

In some cases, the chip or cassette can comprise a gas input line. In some gases, a chip or cassette with a gas input line does not have a gas permeable membrane. A gas input line can allow for gas to be injected into the chip or cassette, thereby dissolving directly onto the fluid within the chip or cassette. In some cases, a gas input is separate from a fluid input. The chip or cassette may also comprise a gas outlet. In some gases, the gas outlet contains a filter to reduce convection and increase gas residence time above the fluid in the chip or cassette, thereby favoring gas diffusion into the fluid.

In some cases, the chip or cassette can comprise a plurality of components or layer comprising a plurality of materials. The plurality of materials can comprise different materials. In some cases, the plurality of materials can comprise a cyclic olefin polymer (COP), a cyclic olefin copolymer (COC), or a polydimethylsiloxane (PDMS) material. In some cases, the plurality of materials can comprise a USP Class VI material such as other medical-grade thermoplastics In some cases, the plurality of materials can comprise any type of material that is biocompatible and/or biostable. In some cases, the materials for the various components or layers of the chip can have a high permeability (e.g., liquid or gas permeability) to permit a flow of fluid and/or cells into, out of, or through the chip (and any components or layers thereof).

The presently disclosed chips can also contain a filter, e.g., filter membrane made of polyethersulfone (PES). The filter can have one or more pores. The pore size can be about 5 μm or less, which can be used to retain cells of 10-μm diameter in the bioprocessing chamber.

### Bubbles and Fluid Priming

In some cases, systems provided herein can be primed with fluid, e.g., in order to facilitate injection of the growth media. This priming can reduce the interfacial tension effects that can be associated with flowing liquid in an initially gas-filled chamber. Interfacial tension between gas-liquid can contribute to hydrodynamic resistance. This can be true of microfluidic devices with relatively small dimensions, whose inherent resistance can be high.

In some cases, the chips and chip arrays (e.g., cassettes) disclosed herein can have a height of the bioprocessing chamber of around 3-5 mm, such that priming is no longer needed, as the fluid does not experience significant resistance when injected into the bioprocessing chamber.

### Evaporation

Due to the fact that PDMS is gas permeable, evaporation can happen, resulting in bubbles in the bioprocessing chamber. In a bioprocessing chamber of small heights, the bubbles can be detrimental to cell growth.

In the presently disclosed chips and chip arrays (e.g., cassettes), the height of the bioprocessing area can be, e.g., around 3-5 mm, which can permit natural “separation” of the cells and occurring bubbles. While the cells settle at the bottom surface, bubbles can be naturally buoyant and thus float towards the top part of the bioprocessing chamber, away from the cells. The environment can be controlled to minimize evaporation and mitigate impacts on the cell growth.

### Seeding

The chips and cassettes provided herein can permit high efficiency cell seeding, while minimizing loss. Such cell seeding may be determined by monitoring the number of cells that are provided to a bioprocessing chamber and the number of cells that adhere to a portion or a surface of the bioprocessing chamber. The cells can be spread homogeneously throughout the bioprocessing chamber to enable optimal growth. In some cases, confluent growth can be prematurely reached in some areas and therefore decrease cell culture efficiency.

The presence of the filter (e.g., filter membrane) can help in blocking cells from prematurely exiting the bioprocessing chamber, ensuring that they stay detained inside the bioprocessing chamber. Mass transport or advection can be a phenomenon due in large part that cells can be relatively large (>10 μm). Their large size can help them sediment into the recess. To help in homogenous distribution, the chip can be attached to a mechanical agitation device, which can facilitate re-distribution of the seeded cells all throughout the bioprocessing chambers. In some cases, the mechanical agitation device can be used with a single, chip, a plurality of chips, a chip array, multiple chip arrays, or any cassettes or compartments of such cassettes.

In some cases, the cells can comprise at least 20,000, 200,000, 350,000, 500,000, 1,000,000, 3,500,000, 10,000,00, 25,000,000, or 50,000,000 cells/mL. In any of the embodiments described herein, the cells can comprise microorganisms, mammalian cells, HEK293 cells, T-cells, Jurkat cells, CHO cells, mesenchymal stem cells, embryonic stem cells, induced pluripotent stem cells, or hematopoietic stem cells. The bioprocessing chambers can comprise at least 0.35, 0.5, 1, 3.5, 5, 10, 15, or 20 million cells/mL.

### Perfusion

Cells can deplete surrounding media from nutrients in static conditions. The rate of media flow in the microfluidic chip can be carefully regulated. The chip designs disclosed herein can balance perfusion flow rate with shear stress so that cells get access to enough nutrients while at the same time, the flow is low enough so as not to remove the cells from their substrate.

In the microscale, numerous parameters can be involved in ensuring cell growth, including temperature gradients, oxygen levels, chemical gradients, cell-to-cell interactions, cell-to-molecule interactions, CO₂ level, shear stress, and cell-substrate interactions.

The fact that the perfusion input channels can be on a different plane than the cells (settling at the bottom of the bioprocessing chamber), means that they can be protected from damaging shear stress induced by the fluid streamlines during perfusion. Shear stress can decrease with depth. Thus, the cells can avoid significant shear stress in the chip/chip array/cassette configurations described herein.

In some cases, the perfusion can come from the width-wise side because one or more fluid inlets are positioned at a higher plane than the bottom of the bioprocessing chamber, which can cause the reduction in shear stress. When injected from the lengthwise side, the input channels can be positioned at a higher plane than the bottom surface of the bioprocessing chamber.

Nutrient and gas diffusion as well as cell consumption can also be optimized. When cells are not homogeneously distributed during seeding, a consumption rate that follows a Poisson distribution can be expected where there is a higher consumption rate near the position of the feeding input channels (since it can be in first contact with the nutrients). Mechanical agitation can be employed during seeding can be beneficial for perfusion to ensure that nutrients are distributed all throughout the bioprocessing chamber. In some embodiments, the system can comprise an agitation device comprising one or more motors configured to produce vibration waveforms. The vibration waveforms may or may not be periodic. The vibration waveforms can agitate at least a portion of one or more chips in a parallelized array, which may aid in homogenously distributing cells or other solid particles in one or more bioprocessing chambers of a parallelized chip array. In some embodiments, a human operator may manually agitate at least a portion of the one or more chips in the parallelized array to aid in homogenously distributing cells or other solid particles in the one or more bioprocessing chambers of the parallelized chip array.

In some embodiments, the methods described herein can further comprise expanding the distributed cells to generate expanded cells. In some cases, the expanding comprises expanding the distributed cells about, or at least 2-fold, 3-fold, 4-fold, 5-fold, 6-fold, 6.5-fold, 7-fold, 8-fold, 9-fold, 10-fold, 11-fold, 12-fold, 25-fold, 50-fold, 100-fold, 150-fold, or 200-fold. In some cases, the expanding occurs over about, or at least 24 hr, 48 h, 72 hr, 96 hr, 120 hr, 144 hr, 168 hr, 192 hr, 216 hr, 240 hr, 264 hr, 288 hr, 312 hr, 336 hr, 360 hr, 720 hr, 1080 hr, or 1200 hr.

### Coating

Appropriate surface treatment can also be performed inside the bioprocessing chamber of the chip or cassette depending on the experimental conditions. Such surface treatments can allow adherent particles or cells to stick unto the surface such that particle-wall adhesion takes place. Additional coating methods can be used to facilitate cell attachment or detachment on the COC substrate. In some cases, the coating can comprise one or more polymeric surfactants. In some cases, the coating can comprise any type of biocompatible or biostable material that facilitates cell adhesion or growth. In some cases, the coating may comprise, for example, biological materials such as extracellular matrix, attachment and adhesion proteins, collagen, laminin, fibronectin, mucopolysaccharides, heparin sulfate, hyaluronidase, or chondroitin sulfate. In some cases, the coating may comprise a non-biological material.

### Harvest

Positioning the harvest/collection channels on a different plane can optimize space by effectively reducing the dead space between chips on a cassette. This can also reduce to constraint of adding lateral channels on either side of the chip in the lengthwise direction, which can make the chip design cumbersome and at the same time, generally adds chip footprint.

In some cases, harvesting can comprise harvesting at least 75%, 80%, 85%, 90%, 95%, 96%, 97%, 98%, or 99% of the cells to provide harvested cells. Harvesting may begin when the seeded or expanded cells are collected by any manual or automatic operation or process, and may end when at least a portion of the seeded or expanded cells are collected by a human or a machine. In some cases, the harvesting occurs in 5 min or less, 1 min or less, 50 seconds or less, 40 seconds or less, 30 seconds or less, 20 seconds or less, 10 seconds or less, or 5 seconds or less. In some cases, at least 75%, 80%, 85%, 90%, 91%, 92%, 93%, or 94% of the harvested cells can be viable. In some cases, the harvesting of cells from the presently disclosed chips can result in about, or least, 99%, 98%, 97%, 96%, 95%, 94%, 93%, 92%, 91%, or 90% cell recovery with about or at least 95%, 94%, 93%, 92%, 91%, or 90% viability for the recovered cells.

Harvesting cells through the bottom surface of the bioprocessing chamber can take advantage of gravity, facilitating the exit of the fluid and cells. Drawing the fluid out (via a syringe pump or negative pressure) while pushing fluid from the perfusion side (via a second syringe pump or positive pressure) can also help complete removal of the fluid and cells to increase harvest efficiency.

### Sampling

Sampling can involve taking representative samples of cells from inside the bioprocessing chamber without interfering with cell growth and without opening the chip. Varying the drawing flow rate at the harvest or collection drain can control the amount of fluid (and cells) collected.

### High Throughput Platforms

The parallelization systems and methods disclosed herein can exhibit the following characteristics:
optimal configuration of space such as arrangement of chips and branching of input and output tubes such that culture space is maximized, e.g., at least 1000 cm² and representing >70% of total chip footprint;
does not involve use of bulky and numerous connections;
does not require high pressures to inject fluid throughout the array (cassette);
design or protocol can be managed in a way that cells do not settle in the input channels but rather in the bioprocessing chamber;
minimization of volume in input channels;
bioprocessing chamber is unobstructed for imaging;
cassette can be manufactured at high volumes and cheaply; chip and cassette can be manufactured via existing (or a modification thereof) manufacturing techniques; and
chips and cassettes can be sterilized using standard methods (e.g., autoclave, gamma irradiation).

### Compartmentalization

As more chips are parallelized, resistances can increase, which can require large pressures to inject fluids into the parallelized chip arrays disclosed herein. FIGS. 11-14 and FIG. 15A show numerical simulations of an increasing array of chips, resulting to a normalized pressure curve as a function of the number of chips. The graph in FIG. 15A shows two slopes, a steep one and a relatively moderate one. The difference can be due the parallelization strategy. When all of the chips in the array are fluidically connected within one layer, channel length can increase in order to reach all the chips on one layer, which can then increase the hydrodynamic resistance and therefore pressure. This can be the case for a standard design that does not comprise or utilize the features described herein.

As more chips are parallelized on a cassette, the entire array can be compartmentalized into smaller arrays. This can make the cross-section that the fluid passes through more manageable because the fluid is no longer required to feed all of the chips on a cassette but only a portion of them. As a consequence of compartmentalization, feeding inputs can be separated into different layers, which can then permit feeding into smaller arrays.

As more chips are being parallelized or multiplexed on a cassette, more feeding input layers can be added to manage the global pressure across the network. In the examples illustrated in FIGS. 11-14, a 16-chip array can be taken as one fluidically connected array on a cassette, which can then be separated into compartments to create 32-chip, 64-chip, and 128-chip arrays. A group of compartments can be referred to herein as a cluster.

In some embodiments, the 16-chip array on a cassette can also be separated into smaller compartments of four separate 4-chip arrays on a cassette. A 16-chip array that has one input and output fluidically connected in the same layer as the chambers can have a large resistance because the fluid needs to travel greater lengths to reach all the chips in the array.

Referring back to FIG. 7, in some cases the 16-chip array can be separated into four smaller arrays consisting of four chips per array. This can effectively create a primary feeding input layer at a layer above the chips. The primary feeding input layer can feed fluid into four compartments. The compartments may not or need not be fluidically connected within the same layer and that the compartments can be fed by different secondary inputs coming from the primary input layer. As a result, as the fluid can reach each compartment with minimal length of travel; thereby providing minimal resistance and thus requiring lower pressures for the feed fluid.

### Embodiments

### Cassette

In one aspect, the present disclosure provides a cassette for bioprocessing. The cassette can comprise a feeding input (201), a branched network of primary feeding input channels in a first plane (204a), and a branched network of primary feeding input channels in a second plane (204b).

In some cases, the cassette can further comprise a bioprocessing layer comprising a plurality of bioprocessing chambers (207). In some embodiments, the plurality of bioprocessing chambers may be fluidically connected to (i) the branched network of primary feeding input channels in the first plane and (ii) the branched network of primary feeding input channels in the second plane. In some cases, the plurality of bioprocessing chambers may be fluidically connected to the branched network of primary feeding input channels in the first plane via the branched network of primary feeding input channels in the second plane.

In some cases, the cassette can further comprise a branched network of primary feeding output channels (203) and a feeding output (214).

In some cases, the cassette can further comprise a plurality of filters (206) (e.g., filter membranes).

In some cases, the cassette can further comprise a collection output (215), a plurality of collection drains (208), a branched network of primary collection channels in a third plane (210a), and a branched network of primary collection channels in a fourth plane (210b). In some embodiments, the first plane, the second plane, the third plane, and/or the fourth plane are substantially parallel.

In some cases, the cassette permits a fluid to flow in a flow path comprising, in order, the feeding input (201), the branched network of primary feeding in-put channels in the first plane (204a), the branched network of primary feeding input channels in the second plane (204b), the plurality of bioprocessing chambers (207), the plurality of filters (e.g., filter membranes), the branched network of primary feeding output channels (203), and the feeding output (214).

In some cases, any one or more of the cassettes disclosed herein can be provided, and a fluid can be flowed along a flow path as described elsewhere herein. In some cases, the fluid can comprise one or more cells.

In some cases, the cells can be deposited into the plurality of bioprocessing chambers (207). In other cases, the cells may not or need not be deposited in the branched network of primary feeding input channels in a first plane (204a) and branched network of primary feeding input channels in a second plane (204b).

In some cases, one or more filters (206) (e.g., filter membranes) can be used to prevent the cells from entering the branched network of primary feeding output channels (203).

In some cases, the cassette can comprise a second flow path comprising, in order, the feeding input (201), the branched network of primary feeding input channels in the first plane (204a), the branched network of primary feeding input channels in the second plane (204b), the plurality of bioprocessing chambers (207), the plurality of collection drains (208), the branched network of primary collection channels in the third plane (210a), the branched network of primary collection channels in the fourth plane (210b), and the collection output (215). In some cases, the plurality of bioprocessing chambers (207) can comprise a plurality of cells. The chambers can comprise at least 0.05 million, 0.25 million, 0.5 million, 1 million, 10 million, 50 million, 100 million, 150 million, 500 million, 1 billion, or 5 billion cells.

In some cases, the cells can be harvested through the plurality of collection drains. In some cases, the harvesting can comprise applying a suction to the second flow path. In some cases, harvesting can comprise harvesting at least 75%, 80%, 85%, 90%, 95%, 96%, 97%, 98%, or 99% of the cells to provide harvested cells. In some cases, the harvesting occurs in 5 min or less, 1 min or less, 50 seconds or less, 40 seconds or less, 30 seconds or less, 20 seconds or less, 10 seconds or less, or 5 seconds or less. In some cases, at least 75%, 80%, 85%, 90%, 91%, 92%, 93%, or 94% of the harvested cells can be viable.

### Multiple Fluid Layer Cassette with Bioprocessing Layer

In one aspect, the present disclosure provides a multiple fluid layer cassette with a bioprocessing layer. The cassette can comprise a branched network of primary feeding input channels in a first plane (204a) and a branched network of primary feeding input channels in a second plane (204b). In some cases, the first plane can be substantially parallel to the second plane.

The cassette can further comprise a bioprocessing layer comprising a plurality of bioprocessing chambers (207) fluidically connected to the branched network of primary feeding input channels in the first plane (204a) and the branched network of primary feeding input channels in the second plane (204b). In some cases, the cassette permits a fluid to flow from the branched network of primary feeding input channels in the first plane (204a), through the branched network of primary feeding input channels in the second plane (204b) and into or through the plurality of bioprocessing chambers (207). In some embodiments, the plurality of bioprocessing chambers (207) can comprise at least 4, 8, 16, 32, 64, 128, or 256 bioprocessing chambers. The plurality of bioprocessing chambers (207) can comprise 2ⁿ bioprocessing chambers, where n is any integer greater than zero. In some embodiments, the plurality of bioprocessing chambers (207) can be fluidically connected to each other in series or in parallel. In some cases, the plurality of bioprocessing chambers (207) can be sterile.

In some cases, each bioprocessing chamber (207) of the plurality of bioprocessing chambers (207) comprises a volume of less than 15mL, 10 mL, 7 mL, 5 mL, 4 mL, 3 mL, 2 mL, 1 mL, or 0.5 mL. In some cases, each bioprocessing chamber (207) of the plurality of bioprocessing chambers (207) comprises a cell culturing surface of less than about 60 cm², 50 cm², 40 cm², 30 cm², 20 cm², 10 cm², 6 cm², 5 cm², or 1 cm².

In some embodiments, the plurality of bioprocessing chambers (207) comprises cells. In some cases, each bioprocessing chamber (207) of the plurality of bioprocessing chambers (207) can comprise at least 0.35, 0.5, 1, 3.5, 5, 10, 15, or 20 million cells/mL. In some embodiments, each bioprocessing chamber (207) of the plurality of bioprocessing chambers (207) comprises at least 0.25, 0.5, or 1 million cells. In some cases, the plurality of bioprocessing chambers (207) comprises at least 0.05 million, 0.25 million, 0.5 million, 1 million, 10 million, 50 million, 100 million, 150 million, 500 million, 1 billion, or 5 billion cells.

In some cases, the plurality of bioprocessing chambers (207) can comprise a biocompatible material. In some cases, the biocompatible material can be a U.S. Pharmacopeia Convention (USP) Class VI material.

In some embodiments, the cassette can further comprise a plurality of connecting input channels (212) connecting the branched network of primary feeding input channels in the first plane (204a) and the branched network of primary feeding input channels in the second plane (204b). In some embodiments, the cassette permits the fluid to flow from a connecting input channel (212) of the plurality of connecting input channels (212) to a subset of the plurality of the bioprocessing chambers (207).

In some embodiments, a length dimension of the connecting input channels (212) can be disposed at an angle (e.g., orthogonal) to a length dimension of the branched network of primary feeding input channels in the first plane (204a).

In some embodiments, the branched network of primary feeding input channels in the first plane (204a) comprises symmetrical branched channels with an axis of symmetry about an upstream-most split point in the branched net-work of primary feeding input channels in the first plane (204a). In some cases, the branched network of primary feeding input channels in the first plane (204a) can comprise at least 1, 2, 3, 4, 5, 6, 7, 8, 9, 10, 20, 30, 40, 50, or 60 split points (213).

In some embodiments, the branched network of primary feeding input channels in a second plane (204b) comprises symmetrical branched channels with an axis of symmetry about an upstream most split point in the branched network of primary feeding input channels in a second plane (204b). In some cases, the branched network of primary feeding input channels in a second plane (204b) can comprise at least 1, 2, 3, 4, 5, 6, 7, 8, 9, 10, 20, 30, 40, 50, or 60 split points (213).

In some cases, the branched network of primary feeding input channels in the first plane (204a) can be positioned above the bioprocessing layer of the cassette. In some cases, the branched network of primary feeding input channels in the first plane (204a) can be positioned below the branched network of primary feeding input channels in the second plane (204b). In some cases, the branched network of primary feeding input channels in the first plane (204a) is above the branched network of primary feeding input channels in the second plane (204b).

In any of the embodiments described herein, the cassette can comprise a branched network of primary feeding in-put channels in a third plane fluidically connected to a branched network of primary feeding input channels in the first plane (204a) and a branched network of primary feeding input channels in the second plane (204b). In some cases, first plane, second plane, and third plane can be substantially parallel. In some cases, the cassette comprises a branched network of primary feeding input channels in at least 2, 3, 4, 5, 6, 7, 8, 9, 10 or more planes, wherein the planes are substantially parallel, and the branched network of primary feeding input channels are fluidically connected.

In any of the embodiments described herein, the feeding layer can comprise a feeding input (201). In some cases, the cassette can permit the fluid to flow from the feeding input (201), through the branched network of primary feeding input channels in the first plane (204a), through the branched network of primary feeding input channels in the second plane (204b) and into or through the plurality of bioprocessing chambers (207).

In some cases, the branched network of primary feeding input channels in the first plane (204a) can comprise binary trees. In some cases, the branched network of primary feeding input channels in the second plane (204b) can comprise binary trees.

In some cases, the cassette further comprises a branched network of secondary feeding input channels (205) fluidically connected to, and between, the primary feeding input channels in the second plane (204b) and the plurality of bioprocessing chambers (207).

In any of the embodiments described herein, the cassette can further comprise a branched network of primary feeding output channels (203). In some cases, the cassette permits the fluid to flow from the branched network of primary feeding input channels in the first plane (204a), the branched network of primary feeding input channels in the second plane (204b) and through the plurality of bioprocessing chambers (207), and the branched network of primary feeding out-put channels (203). In some cases, the cassette comprises a branched network of secondary feeding output channels (202) fluidically connected to, and between, the plurality of bioprocessing chambers (207) and the branched network of primary feeding output channels (203). In some cases, the cassette comprises a branched network of feeding output channels (e.g., primary feeding output channels) in at least 2, 3, 4, 5, 6, 7, 8, 9, 10 or more planes, wherein the planes are substantially parallel, and the branched network of feeding output channels (e.g., primary feeding output channels) are fluidically connected.

In any of the embodiments described herein, the cassette can comprise a plurality of filters (206) (e.g., filter membranes). The cassette can permit the fluid to flow from the branched network of primary feeding input channels in the first plane (204a), through the branched network of primary feeding input channels in the second plane (204b), through the plurality of bioprocessing chambers (207), across the plurality of filters (206), and through the branched network of primary feeding output channels (203). In some embodiments, the cassette comprises a feeding input (201).

In any of the embodiments described herein, the cassette can further comprise a plurality of collection drains (208) fluidically connected to the plurality of bioprocessing chambers (207). The plurality of collection drains can be located below the bioprocessing layer (e.g., when fluid is flowed through the cassette). In some cases, the plurality of collection drains (208) can comprise one or more collection channels. In some cases, the collection layer can further comprise a branched network of primary collection channels in a fourth plane (210a) and a branched net-work of primary collection channels in a fifth plane (210b) fluidically connected to the plurality of collection drains (208). The first plane, the second plane, the third plane, the fourth plane, and/or the fifth plane can be substantially parallel. In some cases, a length dimension of the plurality of collection drains (208) may not or need not be orthogonal to a length dimension of the branched network of primary collection channels in the fifth plane (210b).

In some embodiments, the collection layer can further comprise a branched network of secondary collection channels (209) fluidically connected to, and between, the plurality of bioprocessing chambers (207) and the branched network of primary collection channels in the fifth plane (210b). In some cases, the cassette further comprises a collection hole (211) fluidically connected to the primary collection channels in a fifth plane (210b).

In some cases, the cassette can comprise a material that is transparent or translucent. In some cases, the cassette may not or need not comprise one or more valves.

In some embodiments, the cassette permits a substantially equal pressure drop across each bioprocessing chamber (207) of the plurality of bio-processing chambers (207) when the fluid is flowed from the branched network of primary feeding input channels in the first plane (204a), through the branched net-work of primary feeding input channels in the second plane (204b) and through the plurality of bioprocessing chambers (207). The branched network of primary feeding input channels in the first plane (204a), the branched network of primary feeding in-put channels in the second plane (204b) and the plurality of bioprocessing chambers (207) can be configured to permit a flow of fluid therethrough.

### Multiple Fluid Layer Cassette with Low Inlet Channel Volume

In another aspect, the present disclosure provides a cassette comprising a plurality of cassette structures defining a cassette flow path volume from a feeding input (201) to a feeding output (214). The plurality of cassette structures can comprise the feeding input (201), a branched network of primary feeding input channels in a first plane (204a), a branched network of primary feeding input channels in a second plane (204b), which first plane can be substantially parallel to the second plane, a plurality of bioprocessing chambers (207), a branched network of primary feeding output channels (203), a feeding output (214), and a plurality of feeding input structures defining a feeding input flow path volume from the feeding input (201) to the plurality of bioprocessing chambers (207). In some cases, the plurality of feeding input structures can comprise the feeding input (201), the branched network of primary feeding input channels in the first plane (204a), and the branched network of primary feeding input channels in the second plane (204b). In some embodiments, the feeding input flow path volume can be 30%, 20%, or 10% or less of the cassette flow path volume.

In some cases, the plurality of bioprocessing chambers (207) can comprise at least 4, 8, 16, 32, 64, 128, or 256 bioprocessing chambers (207).

In some embodiments, the plurality of cassette structures can further comprise a branched network of primary feeding input channels in a third plane fluidically connected to the branched network of primary feeding input channels in the first plane (204a) and the branched network of primary feeding input channels in the second plane (204b). In some cases, the branched network of primary feeding input channels in the first plane (204a) and the branched network of primary feeding input channels in the second plane (204b) can comprise one or more microfluidic channels. In some cases, the first plane, second plane, and third plane can be substantially parallel.

### Cassette with Optimal Pressure Drop

In another aspect, the present disclosure provides a cassette with an optimal pressure drop. The cassette can comprise a feeding input (201) and a branched network of primary feeding input channels (204a); a feeding output (214) and a branched network of primary feeding output channels (203); and a bioprocessing layer comprising a plurality of bioprocessing chambers (207). In some cases, the cassette permits a fluid to flow from the feeding input (201), through the branched network of primary feeding input channels (204a), through the plurality of bioprocessing chambers (207), through the branched network of primary feeding output channels (203), and to the feeding output. In some cases, the plurality of bioprocessing chambers (207) comprises at least 32, 64, 128, or 256 bioprocessing chambers (207).

In some cases, the cassette provides a [ΔP_{cassette}/ΔP_{bioprocessing chamber}]/n of less than 0.3, wherein n is greater than 20. In some cases, the cassette provides a ΔP_{cassette}/ΔP_{bioprocessing chamber} with a value equal or lower than the result of (1/22)*n+6, wherein n is the number of bioprocessing chambers. In some cases, n is at least 16, 32, 64, 128, or 256. In some cases, the cassette may provide a ΔP_{cassette}/ΔP_{bioprocessing chamber} with a value equal or lower than the result of (1/a)*n+b, where n is the number of bioprocessing chambers.

### Cassette Integrated into a System

In another aspect, the present disclosure provides a system comprising any one or more of the cassettes described herein and a pump fluidically connected to the one or more cassettes. In some cases, the system can further comprise a reagent vessel fluidically connected to the pump and the cassette. In some cases, the system can further comprise an air vessel comprising CO2 fluidically connected to the reagent vessel. In some cases, a concentration of CO2 in the air vessel can be about 5% or 8% in volume.

In some cases, the system can further comprise a de-bubbler fluidically connected to the reagent vessel and/or to the cassette. In some cases, the reagent vessel can be separated from the de-bubbler by a first valve and the de-bubbler can be separated from the cassette by a second valve.

In some cases, the system can further comprise an output vessel fluidically connected to the one or more cassettes. In some cases, the system can further comprise a flow-through sensor connected to the one or more cassettes. In some cases, the system can further comprise a computer electrically connected to the flow-through sensor. In some cases, the system can further comprise an imaging device. In some cases, the system can further comprise an agitator coupled to the one or more cassettes. In any of the embodiments described herein, the system can be automated (e.g., using on one or more computer processors). In some non-limiting embodiments, the system can be operated manually (e.g., either locally or remotely).

### Methods

In another aspect, the present disclosure provides a method for bioprocessing. The method can comprise providing any one or more of the cassettes described herein. The method can further comprise flowing fluid from the branched network of primary feeding input channels in the first plane (204a), through the branched network of primary feeding input channels in the second plane (204b) and through the plurality of bioprocessing chambers (207). The fluid can comprise a plurality of solid particles. In some cases, a pressure at a fluid input of the cassette can be 3 bar or less, 2 bar or less, 1 bar or less, 0.75 bar or less, 0.5 bar or less, 0.25 bar or less, or 0.1 bar or less.

In some cases, each bioprocessing chamber (207) of the plurality of bioprocessing chambers (207) can comprise a fluid inlet. A flow rate of the fluid at the inlet of each bioprocessing chamber of the plurality of bioprocessing chambers (207) can range from about 10 μL/hr×a number of the plurality of bioprocessing chambers (207) to about 10 mL/min×a number of the plurality of bioprocessing chambers (207).

In some cases, a flow rate of the fluid comprising the plurality of solid particles through the branched network of primary feeding input channels in the first plane (204a) and the branched network of primary feeding input channels in the second plane (204b) can be greater than a settling or sedimentation velocity of the plurality of solid particles.

In some embodiments, the solid particles can comprise cells. In some cases, the solid particles can comprise at least 20,000, 200,000, 350,000, 500,000, 1,000,000, 3,500,000, 10,000,00, 25,000,000, or 50,000,000 cells/mL. In any of the embodiments described herein, the cells can comprise microorganisms, mammalian cells, HEK293 cells, T-cells, Jurkat cells, CHO cells, mesenchymal stem cells, embryonic stem cells, induced pluripotent stem cells, or hematopoietic stem cells.

In some embodiments, the method can further comprise distributing a substantially equal amount of the cells to each bioprocessing chamber (207) of the plurality of bioprocessing chambers (207) to provide distributed cells. In some cases, the distributing can occur within 5 minutes.

In some embodiments, the method can further comprise washing the distributed cells. In some embodiments, the method can further comprise expanding the distributed cells to generate expanded cells. In some cases, the expanding comprises expanding the distributed cells about, or at least 2-fold, 3-fold, 4-fold, 5-fold, 6-fold, 6.5-fold, 7-fold, 8-fold, 9-fold, 10-fold, 11-fold, 12-fold, 25-fold, 50-fold, 100-fold, 150-fold, or 200-fold. In some cases, the expanding occurs over about, or at least 24 hr, 48 h, 72 hr, 96 hr, 120 hr, 144 hr, 168 hr, 192 hr, 216 hr, 240 hr, 264 hr, 288 hr, 312 hr, 336 hr, 360 hr, 720 hr, 1080 hr, or 1200 hr.

In some embodiments, the method can further comprise imaging the distributed cells. In some embodiments, the method can further comprise imaging the expanded cells. In some embodiments, the method can further comprise using a computer system to predict time to confluence of the expanded cells.

In some embodiments, the method can further comprise contacting the distributed cells with a reagent after the washing. In some embodiments, the method can further comprise, after the contacting, performing a second wash of the distributed cells. In some embodiments, the method can further comprise, after the second wash of the distributed cells, harvesting the distributed cells.

The reagents can comprise, for example, balanced salt solutions, buffers, detergents, chelators, or any materials or substances that promote or facilitate cell adhesion.

In some cases, the cells can be seeded at a flow rate ranging from 0.1 microliters per second (μL/s) to 10 μL/s or more. In some cases, the cells can be seeded at a flow rate of at least about 0.17 μL/s.

In some cases, the cells can be harvested at a flow rate ranging from about 0.1 microliters per second (μL/s) to about 10 μL/s or more. In some cases, the cells can be harvest at a flow rate ranging from about 0.17 μL/s to about 1.59 μL/s.

In another aspect, the present disclosure provides a method for culturing one or more cells. The method may comprise distributing the one or more cells in one or more bioprocessing chambers. In some embodiments, one or more bottom surfaces of the bioprocessing chambers may be used for cell culturing. The one or more bioprocessing chambers may be associated with one or more cassettes and/or one or more chips within a parallelized array of chips. In some cases, the one or more cells may be cultured using a microfluidic system comprising a plurality of chips arranged in a parallelized configuration. In some cases, the microfluidic system may comprise a plurality of components that are disposed on different layers or planes to enhance fluid flow characteristics through the microfluidic system, perfusion of growth media through the microfluidic system, cell seeding efficiency, and cell harvesting efficiency.

In some embodiments, the method may comprise washing the one or more cells. In some cases, the one or more cells may be washed a plurality of times. In some cases, the one or more cells may be washed using a buffer (e.g., a PBS buffer). The buffer may be provided via one or more perfusion inlets or feeding inputs of the microfluidic system.

In some embodiments, the method may comprise expanding the one or more cells. In some cases, the cells may undergo an expansion that is about, or at least 2-fold, 3-fold, 4-fold, 5-fold, 6-fold, 6.5-fold, 7-fold, 8-fold, 9-fold, 10-fold, 11-fold, 12-fold, 25-fold, 50-fold, 100-fold, 150-fold, or 200-fold. In some cases, the expansion may occur within a time period of at least about 24 hr, 48 hr, 72 hr, 96 hr, 120 hr, 144 hr, 168 hr, 192 hr, 216 hr, 240 hr, 264 hr, 288 hr, 312 hr, 336 hr, 360 hr, 720 hr, 1080 hr, or 1200 hr.

In some embodiments, the method may comprise contacting the one or more cells with at least one reagent. The at least one reagent may comprise, for example, a balanced salt solution, a buffer, a detergent, a chelator, or any materials or substances that promotes or facilitates cell adhesion. In some cases, the reagent may be provided to the one or more cells after the cells have been washed.

In some embodiments, the method may comprise imaging the one or more cells. In some cases, the one or more cells may be imaged using an imaging sensor or a camera. The imaging sensor or camera may be positioned and/or oriented such that an optical/imaging axis of the imaging sensor or camera is aligned with a portion of the microfluidic system comprising the one or more cells. In some cases, the portion of the microfluidic system may be transparent or semi-transparent to facilitate the imaging of the one or more cells.

In some embodiments, the method may comprise harvesting the one or more cells. In some cases, the cells may be harvested via one or more drains. In some cases, the one or more drains may be located on a bottom surface of the plurality of bioprocessing chambers of the microfluidic system.

In another aspect, the present disclosure provides a microfluidic system. The microfluidic system may be configured for cell culturing. The microfluidic system may comprise one or more cassettes or one or more chips arranged in a parallelized configuration as described elsewhere herein.

In some embodiments, the microfluidic system may be configured for flowing a fluid through a fluid input of the microfluidic system at a target pressure. In some cases, the target pressure may be less than 3 bars. In some cases, the target pressure may be less than 2 bars. In some cases, the target pressure may be less than 1 bar. In some cases, the target pressure may be less than 0.5 bars.

In some embodiments, the microfluidic system may be configured for culturing a plurality of cells. In some cases, the plurality of cells may comprise at least about 50 million cells. In some cases, the plurality of cells may comprise at least about 500 million cells. In some cases, the plurality of cells may comprise at least about 1 billion cells.

### Computer Systems

In an aspect, the present disclosure provides computer systems that are programmed or otherwise configured to implement methods of the disclosure, e.g., any of the subject methods for bioprocessing. FIG. 20 shows a computer system 2001 that is programmed or otherwise configured to implement a method for bioprocessing. The computer system 2001 can be configured to, for example, control a flow of fluid comprising one or more cells into or through one or more chips or cassettes. The one or more chips or cassettes can be arranged in a parallelized configuration as described elsewhere herein. In some cases, the computer system 2001 can be configured to adjust a flow rate or an amount of fluid flow into or through the one or more chips or cassettes, based on one or more sensor readings. The computer system 2001 can be further configured to adjust the flow rate or an amount of fluid flow into or through the one or more chips or cassettes in order to optimize (i.e., decrease) an amount of pressure drop across the system. The computer system 2001 can be an electronic device of a user or a computer system that is remotely located with respect to the electronic device. The electronic device can be a mobile electronic device.

The computer system 2001 can include a central processing unit (CPU, also “processor” and “computer processor” herein) 2005, which can be a single core or multi core processor, or a plurality of processors for parallel processing. The computer system 2001 also includes memory or memory location 2010 (e.g., random-access memory, read-only memory, flash memory), electronic storage unit 2015 (e.g., hard disk), communication interface 2020 (e.g., network adapter) for communicating with one or more other systems, and peripheral devices 2025, such as cache, other memory, data storage and/or electronic display adapters. The memory 2010, storage unit 2015, interface 2020 and peripheral devices 2025 are in communication with the CPU 2005 through a communication bus (solid lines), such as a motherboard. The storage unit 2015 can be a data storage unit (or data repository) for storing data. The computer system 2001 can be operatively coupled to a computer network (“network”) 2030 with the aid of the communication interface 2020. The network 2030 can be the Internet, an internet and/or extranet, or an intranet and/or extranet that is in communication with the Internet. The network 2030 in some cases is a telecommunication and/or data network. The network 2030 can include one or more computer servers, which can enable distributed computing, such as cloud computing. The network 2030, in some cases with the aid of the computer system 2001, can implement a peer-to-peer network, which can enable devices coupled to the computer system 2001 to behave as a client or a server.

The CPU 2005 can execute a sequence of machine-readable instructions, which can be embodied in a program or software. The instructions can be stored in a memory location, such as the memory 2010. The instructions can be directed to the CPU 2005, which can subsequently program or otherwise configure the CPU 2005 to implement methods of the present disclosure. Examples of operations performed by the CPU 2005 can include fetch, decode, execute, and writeback.

The CPU 2005 can be part of a circuit, such as an integrated circuit. One or more other components of the system 2001 can be included in the circuit. In some cases, the circuit is an application specific integrated circuit (ASIC).

The storage unit 2015 can store files, such as drivers, libraries and saved programs. The storage unit 2015 can store user data, e.g., user preferences and user programs. The computer system 2001 in some cases can include one or more additional data storage units that are located external to the computer system 2001 (e.g., on a remote server that is in communication with the computer system 2001 through an intranet or the Internet).

The computer system 2001 can communicate with one or more remote computer systems through the network 2030. For instance, the computer system 2001 can communicate with a remote computer system of a user (e.g., an operator managing or monitoring the bioprocessing). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 2001 via the network 2030.

Methods as described herein can be implemented by way of machine (e.g., computer processor) executable code stored on an electronic storage location of the computer system 2001, such as, for example, on the memory 2010 or electronic storage unit 2015. The machine executable or machine-readable code can be provided in the form of software. During use, the code can be executed by the processor 2005. In some cases, the code can be retrieved from the storage unit 2015 and stored on the memory 2010 for ready access by the processor 2005. In some situations, the electronic storage unit 2015 can be precluded, and machine-executable instructions are stored on memory 2010.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 2001, can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which can provide non-transitory storage at any time for the software programming. All or portions of the software can at times be communicated through the Internet or various other telecommunication networks. Such communications, for example, can enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, can take many forms, including but not limited to, a tangible storage medium, a carrier wave medium or physical transmission medium. Non-volatile storage media including, for example, optical or magnetic disks, or any storage devices in any computer(s) or the like, can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media can be involved in carrying one or more sequences of one or more instructions to a processor for execution.

The computer system 2001 can include or be in communication with an electronic display 2035 that comprises a user interface (UI) 2040 for providing, for example, a portal for an operator to monitor or track one or more steps or operations of the bioprocessing methods and systems described herein. The portal can be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2005. For example, the algorithm can be configured to adjust a flow rate or an amount of fluid flow into or through the one or more chips or cassettes, based on one or more sensor readings. In some embodiments, the algorithm can be further configured to adjust the flow rate or an amount of fluid flow into or through the one or more chips or cassettes in order to optimize (i.e., decrease) an amount of pressure drop across the system.

### EXAMPLES

### Example 1

A sample of T-cells is taken from a cancer patient. The T-cells are modified by transduction in a chip to produce chimeric antigen receptors that target the patient's cancer. The cassette of FIG. 21, 22, 23, 24, or 25A is provided, and a cell culture medium comprising the T-cells is flowed from the feeding input (201), through the primary feeding input channel in a first plane (204a), through the connecting input channel (212), through the primary feeding input channel in a second plane (204b), and into the bioprocessing chambers (207). The T-cells are retained in the bioprocessing chambers (207), in part because of the filters (206), while the cell culture medium fluid flows through an opening in the ceiling (1004) of the bioprocessing chambers (207), across the filters (206), and out the secondary feeding output channel (202) and the primary feeding output channel (203) and through the feeding output (214). During the introduction of the T-cells into the bioprocessing chambers (207), the secondary collection channels (209) are filled with fluid and do not permit the cell culture medium to pass through. The drain is primed with fluid and the valve is closed such that there is no fluid flow through the drain. The cassette is agitated on a shaker, and the T-cells in the bioprocessing chambers (207) are allowed to homogenously seed on the bottom surface (1001). After seeding and before proliferation, T-cells are activated by flowing an activation agent through the perfusion inlet. The reagent is left to react with the cells for a certain amount of time. The T-cells are subsequently washed with a washing reagent (e.g., PBS) via the perfusion inlet. The T-cells are subsequently transduced via a viral vector (e.g., lentivirus) via the perfusion inlet. The reagent is left to react with the cells for a certain amount of time. The T-cells are subsequently washed with a washing reagent (e.g., PBS) via the perfusion inlet. Agitation can be done at any time to re-homogenize the cells across the chip surface.

Perfusion is performed to introduce media; the perfusion does not disturb the seeded modified T-cells because they are in the recess and on the bottom surface (1001) (see FIG. 1). The modified T-cells are expanded on the bottom surface for 72 hours. A wash fluid is then provided through the feeding input (201) while suction is applied through the secondary collection channels (209) to collect the expanded modified T-cells. After the wash and before the harvest, the cells are mixed with a formulation reagent (e.g., a cryopreservant) via the perfusion inlet. The cells are then harvested via the harvest drains using the push and pull methods described herein. Enzymes may not or need not be required to facilitate harvesting. Agitation can be used to further increase harvesting efficiency. The collected, expanded modified T-cells are then introduced into the patient to treat the patient's cancer. While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein can be employed in practicing the disclosure. It is therefore contemplated that the disclosure shall also cover any such alternatives, modifications, variations or equivalents. It is intended that the following claims define the scope of the invention and that methods and structures within the scope of these claims and their equivalents be covered thereby.

1.-141. (canceled)

142. A method for bioprocessing, the method comprising:
(a) providing a cassette, comprising:
a bioprocessing chamber comprising a bottom surface;
a branched network of feeding input channels fluidically connected to the bioprocessing chamber at a first end of the bioprocessing chamber;
a plurality of output channels fluidically connected to the bioprocessing chamber at a second end of the bioprocessing chamber opposite the first end, wherein the plurality of output channels are substantially orthogonal to the bottom surface; and
a gas input channel fluidically connected to the bioprocessing chamber;
(b) introducing a fluid and one or more cells into the bioprocessing chamber through the branched network of feeding input channels; and
(c) flowing at least a portion of the fluid from the bioprocessing chamber into the plurality of output channels.

143. The method of claim 142, wherein the gas input channel is substantially orthogonal to the bottom surface.

144. The method of claim 142, wherein a feeding input channel of the branched network of feeding input channels is disposed across the bioprocessing chamber from an output channel of the plurality of output channels.

145. The method of claim 142, wherein the branched network of feeding input channels comprises symmetrical branched channels with an axis of symmetry about an upstream-most split point.

146. The method of claim 142, wherein the branched network of feeding input channels comprises binary trees.

147. The method of claim 142, wherein the bioprocessing chamber comprises a curved edge at an end of bioprocessing chamber.

148. The method of claim 142, wherein the solid support comprises an optically transparent material.

149. The method of claim 142, wherein the bioprocessing chamber comprises one or more walls, wherein the one or more walls, the bottom surface, or both, comprise cyclic olefin copolymer (COC).

150. The method of claim 142, further comprising a ceiling, wherein the ceiling is disposed across the bioprocessing chamber opposite of the bottom surface.

151. The method of claim 150, wherein the ceiling comprises a gas permeable material.

152. The method of claim 150, wherein a height of the bioprocessing chamber is at least 0.5 mm, and wherein the height of the bioprocessing chamber is measured between the bottom surface and the ceiling.

153. The method of claim 142, wherein the bioprocessing chamber is treated with a coating, and wherein the coating interacts with or adheres to the bottom surface via curing, covalent bonding, or incubation.

154. The method of claim 150, wherein at least a portion of an output channel of the plurality of output channels extends into the bioprocessing chamber.

155. The method of claim 154, wherein the output channel extends from the ceiling into the bioprocessing chamber.

156. The method of claim 150, further comprising additional gas input channels fluidically connected to the bioprocessing chamber.

157. The method of claim 156, wherein the gas input channel is fluidically connected to the bioprocessing chamber via the ceiling.

158. The method of claim 150, wherein the output channel is fluidically connected to the bioprocessing chamber via the ceiling at the first end of the bioprocessing chamber.

159. The method of claim 152, wherein a height of the branched network of feeding input channels is less than the height of the bioprocessing chamber.

160. The method of claim 142, wherein the fluid is configured to enter the cassette through the branched network of feeding input channels, flow through the bioprocessing chamber, and exit the cassette through the plurality of output channels.

161. The method of claim 142, wherein during (c), at least 90% of the one or more cells remain in the bioprocessing chamber.

162. The method of claim 142, further comprising expanding the one or more cells in the bioprocessing chamber, wherein the expanding comprises expanding the one or more cells at least 50-fold, 100-fold, 150-fold, or 200-fold.

163. The method of claim 142, further comprising culturing the one or more cells in the bioprocessing chamber at greater than about 90% cell seeding efficiency.

164. The method of claim 142, wherein the flowing the at least a portion of the fluid comprises flowing the at least a portion of the fluid at a pressure of less than about 3 bar.

165. The method of claim 142, further comprising harvesting at least about 90% of the one or more cells to yield recovered cells, wherein at least about 90% of the recovered cells are viable.

166. The method of claim 165, wherein the one or more cells are harvested at a flow rate of greater than 10 microliters/second (μL/s).

167. A cassette, comprising:
a bioprocessing chamber comprising a bottom surface;
a branched network of feeding input channels fluidically connected to the bioprocessing chamber at a first end of the bioprocessing chamber;
a plurality of output channels fluidically connected to the bioprocessing chamber at a second end of the bioprocessing chamber opposite the first end, wherein the output channel is substantially orthogonal to the bottom surface; and
a gas input channel fluidically connected to the bioprocessing chamber.
